(19) United States
(12) Patent Application Publication
(10) Pub. No.: US 2026/0297014 A1
(43) Pub. Date: Oct. 1, 2026

(54) Reactive Separation Systems for Recovery or Valorization of Carboxylic Acids from Dilute Streams

(71) Applicant: Dioxycle, Saint Ouen sur Seine (FR)

(72) Inventors: Jonathan Maistrello, La Garenne Colombes (FR); Raphaël Sanjuan, St Maur des Fossés (FR); Paul S. Wallace, Paris (FR); Georges Matta, La Garenne-Colombes (FR)

(21) Appl. No.: 19/630,401

(22) Filed: Mar. 26, 2026

Related U.S. Application Data
(60) Provisional application No. 63/780,161, filed on Mar. 28, 2025.
(60) Provisional application No. 63/889,191, filed on Sep. 26, 2025.

(51) Int. Cl. C07C 67/60; B01D 3/00; B01D 3/14; C25B 15/08

(52) U.S. Cl. CPC C07C 67/60; B01D 3/009; B01D 3/143; C25B 15/081

(57) ABSTRACT

This disclosure relates to systems and methods for producing purified chemical products from aqueous carboxylic acids. A disclosed method includes introducing carboxylic acid and alcohol streams into a reactive distillation esterification column, reacting the carboxylic acid and alcohol to form an ester, removing the ester stream from the column while it is reacting, and processing the ester stream in a downstream purification system to purify the product.

### CROSS REFERENCE TO RELATED APPLICATIONS

This application claims the benefit of U.S. Provisional Pat. App. No. 63/889,191, filed Sep. 26, 2025, and U.S. Provisional Pat. App. No. 63/780,161, filed Mar. 28, 2025, which are both incorporated by reference herein in their entireties for all purposes.

### BACKGROUND

In order to combat global warming, there is an urgent need to displace virgin-fossil-resource-based fuels and chemicals with low-carbon-intensity fuels and chemicals. Producing chemicals from hydrocarbons without releasing carbon dioxide (CO₂) emissions through strategies such as electrification of heat production to replace traditional gas-fired heaters is an area of technology being investigated. Producing chemicals and fuels from the recycling of carbon dioxide is another area of technology currently being investigated. This second strategy presents the dual environmental benefit of capturing carbon emissions from a first process into a chemical while also avoiding the emission of conventional fossil fuels usually employed to produce such chemicals. Carbon oxide electrolyzers that can transform CO₂ or carbon monoxide (CO) into useful chemicals are technically feasible but require energy efficiency improvements to lower operational and capital costs enough to make the generated fuels and chemicals cost competitive with fossil sources.

One target for carbon oxide conversion comprises carboxylic acids, such as formic acid, acetic acid, and other short-chain carboxylic acids, which are widely used as chemical intermediates, solvents, and feedstocks for industrial processes. These compounds can be produced in other ways such as catalytic, biological, and electrochemical processes. In many emerging production technologies, particularly electrochemical carbon oxide conversion processes, the resulting product streams contain relatively dilute concentrations of carboxylic acids in aqueous solution. For example, electrochemical reduction of carbon dioxide or carbon monoxide can generate aqueous streams containing carboxylic acids or corresponding carboxylate species that may be present at concentrations of less than about 20 wt %. Purification of carboxylic acids from dilute aqueous streams can present significant challenges. Conventional separation techniques such as distillation, extraction, or membrane separation can become too energy intensive to be economically viable when the acid concentration is low or when the boiling points of the acid and water are similar, and it is often not cost effective to purify these aqueous streams. In addition, such streams may contain dissolved salts, electrolytes, or other impurities that complicate downstream purification.

### SUMMARY

This disclosure relates to systems and methods for producing purified chemical products, for example carboxylic acids or esters, from dilute aqueous streams containing carboxylic acids. The present disclosure provides for systems where a dilute aqueous stream containing a carboxylic acid is supplied to a reactive distillation esterification column together with an alcohol. Within the reactive distillation column, the carboxylic acid reacts with the alcohol to form a corresponding ester. Carboxylic acids often have similar boiling points to water, which make conventional distillation methods difficult, particularly when the acid and water form azeotropes. The ester form is more readily separable and can be removed as a top product from the column as an ester-containing vapor stream as the ester is formed. Removal of the ester from the reaction environment can shift the reaction equilibrium and facilitate recovery of products derived from dilute carboxylic acid streams. This separation and purification can be accomplished at lower power than similar conventional methods, especially since some energy can be reused in other parts of the system. One example is waste heat from electrolyzers being used in distillation for low boiling point esters.

In certain embodiments, the ester-containing stream can be supplied to a downstream separation system configured to produce a purified chemical product. In specific embodiments, the purified chemical product is a purified ester product, but in other embodiments, the ester can be hydrolyzed to regenerate the corresponding carboxylic acid and alcohol in purified form. Regenerated alcohol can be recycled to the reactive distillation esterification column, thereby reducing or eliminating consumption of makeup alcohol and enabling efficient purification of dilute carboxylic acid streams.

In specific embodiments, the dilute carboxylic acid stream supplied to the reactive distillation column can be produced by a carbon oxide electrolyzer system configured to convert carbon monoxide or carbon dioxide into carboxylic acids or corresponding carboxylate species. In specific embodiments, the reactive distillation esterification column can also facilitate separation of non-volatile components present in the feed stream, for example, water, salts, electrolytes, or other impurities that can interfere with other purification methods. Some salts, if allowed to remain in the esterification column, can affect the pH and can have an interfering catalytic effect on the esterification reaction. In specific embodiments, the aqueous feed stream contains relatively low concentrations of the carboxylic acid, for example less than about 20 wt %. Conversion of the carboxylic acid to a corresponding ester within the reactive distillation column can facilitate separation of the acid from water and other non-volatile components, even at very low concentrations.

In specific embodiments of the invention, a method is provided. The method includes introducing an aqueous feed stream containing carboxylic acid into a reactive distillation esterification column, introducing an alcohol stream into the reactive distillation esterification column, and esterifying the carboxylic acid with the alcohol within the reactive distillation esterification column to form an ester. The method also includes steps of removing an ester-containing vapor stream from the reactive distillation esterification column as the ester is formed and processing the ester-containing vapor stream in a downstream purification system to produce the purified chemical product.

In specific embodiments of the invention, a system is provided. The system includes a reactive distillation esterification column that has an aqueous feed inlet configured to receive an aqueous stream comprising a carboxylic acid, an alcohol inlet configured to receive an alcohol, a reaction zone within the column in which the carboxylic acid reacts with the alcohol to form an ester, an overhead vapor outlet configured to withdraw an ester-containing vapor stream, and a bottoms outlet configured to discharge a bottoms stream. The system also includes a downstream purification system fluidly connected to the overhead vapor outlet that receives the ester-containing vapor stream and produces the purified chemical product.

In specific embodiments of the invention, a method is provided. The method includes producing an ester-containing stream by reacting an aqueous carboxylic acid stream with an alcohol stream in a reactive distillation esterification column, hydrolyzing at least a portion of the ester-containing stream to produce a hydrolysis stream comprising the carboxylic acid and the alcohol, recovering a carboxylic acid product stream having a greater concentration than the aqueous carboxylic acid stream, and recycling at least a portion of the alcohol produced during hydrolysis to the reactive distillation esterification column.

### BRIEF DESCRIPTION OF THE DRAWINGS

The accompanying drawings illustrate various embodiments of systems, methods, and various other aspects of the disclosure. A person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 provides a diagram of a system for purification or valorization of dilute carboxylic acids in accordance with specific embodiments of the inventions disclosed herein.

FIG. 2 provides a diagram of a system for purification of a carboxylic acid in accordance with specific embodiments of the inventions disclosed herein.

FIG. 3 provides a diagram of a system for producing a purified ester product in accordance with specific embodiments of the inventions disclosed herein.

FIG. 4 provides a diagram of an acidic electrolyzer system in accordance with specific embodiments of the inventions disclosed herein.

FIG. 5 provides a diagram of an alkaline electrolyzer system in accordance with specific embodiments of the inventions disclosed herein.

FIG. 6 provides a diagram of a single-column reactive distillation esterification system in accordance with specific embodiments of the inventions disclosed herein.

FIG. 7 provides a diagram of a multi-column reactive distillation esterification system in accordance with specific embodiments of the inventions disclosed herein.

FIG. 8 provides a diagrams of hydrolysis systems in accordance with specific embodiments of the inventions disclosed herein.

FIG. 9 provides a diagram of an acid drying system in accordance with specific embodiments of the inventions disclosed herein.

FIG. 10 provides a diagram of an ester/alcohol separation system in accordance with specific embodiments of the inventions disclosed herein.

FIG. 11 provides a diagram of a system for recovering formic acid from an electrolyzer stream using reactive distillation esterification and a hydrolysis reactor in accordance with specific embodiments of the inventions disclosed herein.

FIG. 12 provides a diagram of a system for recovering formic acid from an electrolyzer stream using reactive distillation esterification and reactive distillation hydrolysis in accordance with specific embodiments of the inventions disclosed herein.

FIG. 13 provides a diagram of a system for recovering acetic acid from an electrolyzer stream using reactive distillation esterification and a hydrolysis reactor in accordance with specific embodiments of the inventions disclosed herein.

FIG. 14 provides a diagram of a system for recovering acetic acid from an electrolyzer stream using reactive distillation esterification and an integrated hydrolysis and separation system in accordance with specific embodiments of the inventions disclosed herein.

FIG. 15 provides a diagram of a system for producing an ester product from a dilute carboxylic acid stream using reactive distillation esterification and downstream ester purification in accordance with specific embodiments of the inventions disclosed herein.

FIG. 16 provides a diagram of a process for producing a purified chemical product in accordance with specific embodiments of the inventions disclosed herein.

FIG. 17 provides a diagram of a process for purifying a carboxylic acid stream in accordance with specific embodiments of the inventions disclosed herein.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Different systems and methods for carboxylic acid recovery, valorization, or purification are described in detail in this disclosure. The methods and systems disclosed in this section are nonlimiting embodiments of the invention, are provided for explanatory purposes only, and should not be used to constrict the full scope of the invention. It is to be understood that the disclosed embodiments may or may not overlap with each other. Thus, part of one embodiment, or specific embodiments thereof, may or may not fall within the ambit of another, or specific embodiments thereof, and vice versa. Different embodiments from different aspects may be combined or practiced separately. Many different combinations and sub-combinations of the representative embodiments shown within the broad framework of this invention, that may be apparent to those skilled in the art but not explicitly shown or described, should not be construed as precluded.

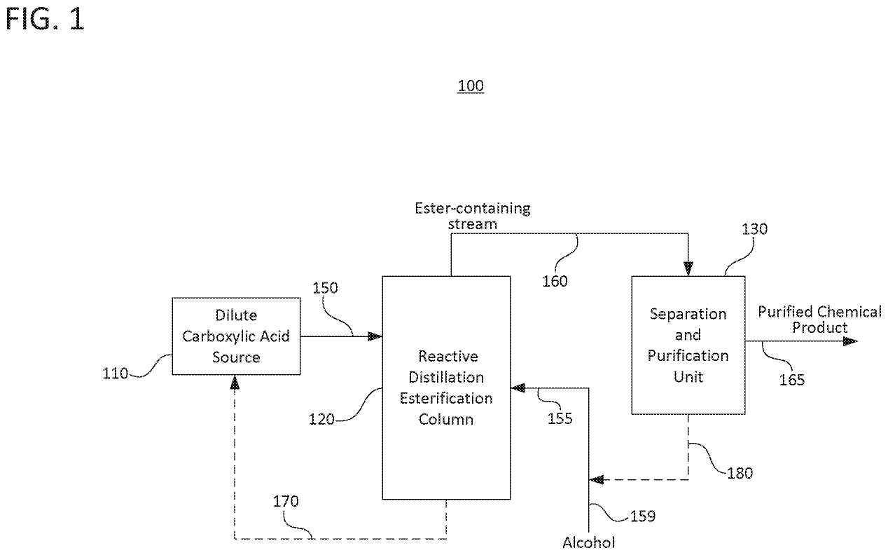

FIG. 1 shows a diagram of a system 100 for purification or valorization of dilute carboxylic acid in accordance with specific embodiments of the inventions disclosed herein. Systems and methods as described herein enable recovery or conversion of carboxylic acids present in dilute aqueous streams by converting the carboxylic acid to a corresponding ester using reactive distillation and subsequently processing the ester to produce a desired product. In specific embodiments, the ester may be further processed to regenerate the corresponding carboxylic acid. In specific embodiments, the ester may be produced as a purified ester product.

In the embodiment illustrated in FIG. 1, a dilute carboxylic acid source 110 supplies an aqueous feed stream 150 containing a carboxylic acid. The dilute carboxylic acid source 110 may correspond to any process that produces an aqueous stream containing a carboxylic acid. For example, such streams may be produced by carbon oxide electrolyzers, catalytic oxidation, biological fermentation, absorption scrubbing systems, or other industrial processes that generate carboxylic acids in aqueous media. In many such processes, the carboxylic acid is present at relatively low concentrations, for example less than about 20 wt %, which can make direct purification by conventional distillation, extractive distillation, or solvent extraction difficult, especially when the boiling points of the carboxylic acid and water are relatively near to each other.

The aqueous feed stream 150 is supplied to a reactive distillation esterification column 120 together with an alcohol stream 155. In specific embodiments, the alcohol stream is a feed stream 159 supplied externally as a reagent. In specific embodiments, part or all of the alcohol stream can be supplied by a downstream unit in the system 100. Within the reactive distillation esterification column 120, the carboxylic acid reacts with the alcohol to form a corresponding ester. The reactive distillation column simultaneously promotes the esterification reaction and separates the ester from the reaction mixture by vapor-liquid separation.

In specific embodiments, the dilute carboxylic acid source can contain more than one carboxylic acid (e.g., acetic acid and propionic acid). In this case, the alcohol stream fed into the esterification column can react to form two different esters.

In specific embodiments, the ester produced in the reactive distillation column may be removed from the column in an ester-containing vapor stream 160 as the ester is formed. Removal of the ester from the reaction environment may shift the reaction equilibrium and promote continued conversion of the carboxylic acid to the ester. Removal of water from the reactive zone of the column 120 would similarly favor conversion to the ester. For example, if the carboxylic acid is formic acid and the alcohol is methanol, then the esterification reaction to methyl formate is shown in Eq. 1 as:

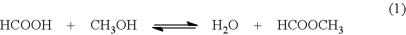

In addition, non-volatile components of the feed stream, including water, dissolved salts, electrolytes, or other impurities, may remain in a bottoms stream 170 discharged from the reactive distillation column. In the example shown here, the bottoms stream is optionally routed to be reused in the dilute carboxylic acid source 110, but in other embodiments, part or all of the bottoms stream 170 could be used in other portions of the system. The ester-containing vapor stream 160 may be supplied to a downstream separation and purification system 130, which outputs a purified chemical product 165. In specific embodiments, the purified chemical product 165 is a purified ester product. In other embodiments, the purified chemical product 165 is the carboxylic acid regenerated through hydrolysis. Alcohol separated in the downstream separation and purification system can be optionally recycled to the reactive distillation column through one or more recycled alcohol streams 180. In specific embodiments, input stream 155 can be partly or wholly from recycled alcohol stream 180 or alternatively combined with alcohol stream 159, which can act as a feed stream or as a makeup stream to account for losses in the system.

The systems described herein may therefore enable purification or concentration of carboxylic acids from dilute aqueous streams by converting the carboxylic acid to a corresponding ester that is more readily separable from water and other non-volatile components. The ester may then be further processed to produce either a purified ester product or to regenerate the carboxylic acid product, depending on the desired outcome of the process.

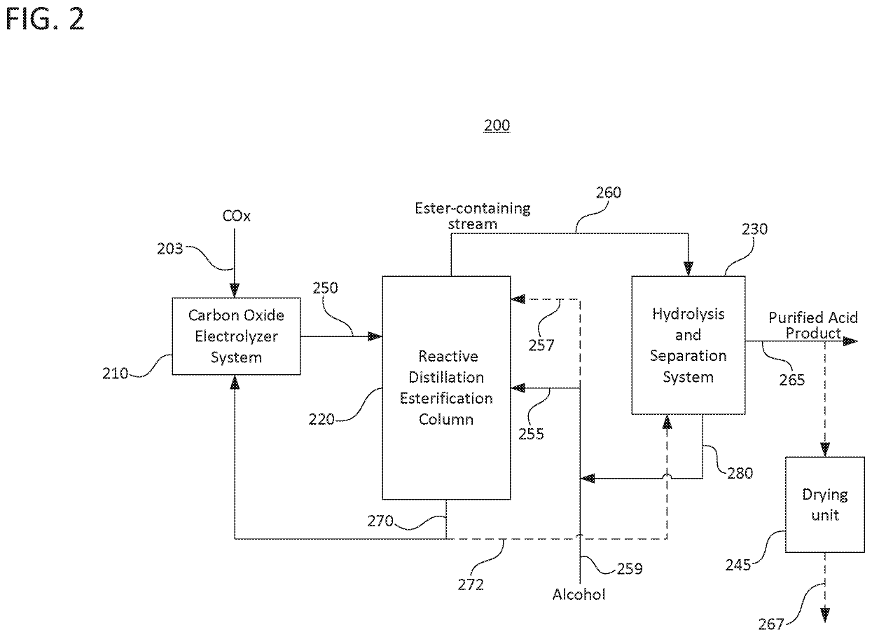

In specific embodiments, the separation and purification system 130 can recover the original carboxylic acid in purer and more concentrated form. One example of this is shown in FIG. 2, which shows a diagram of a system 200 for purification of a carboxylic acid in accordance with specific embodiments of the inventions disclosed herein. System 200 includes a carbon oxide electrolyzer system 210, which represents one example of the dilute carboxylic acid source 110 shown in FIG. 1. Though an electrolyzer is shown in this example, any of the other dilute carboxylic acids discussed in regard to system 100 of FIG. 1 could be used. In this example, the carbon oxide electrolyzer system 210 produces a dilute aqueous carboxylic acid stream 250 using a carbon oxide source 203 such as carbon monoxide (CO) or carbon dioxide (CO₂). The dilute carboxylic acid source in FIG. 2 is not limited to electrolyzer outputs but can have various sources. System 200 also includes a reactive distillation esterification column 220 that converts the carboxylic acid to a corresponding ester, and a hydrolysis and separation system 230 that converts the ester back to the carboxylic acid and separates it into constituents.

In the embodiment shown in FIG. 2, the carbon oxide electrolyzer system 210 produces an aqueous carboxylic acid stream 250 that is supplied to the reactive distillation esterification column 220. Carbon oxide electrolyzer systems can convert carbon monoxide or carbon dioxide to one or more carboxylic acids or corresponding carboxylate species. The aqueous stream produced by the electrolyzer can contain relatively dilute concentrations of the carboxylic acid in water. For example, in specific embodiments, the carboxylic acid concentration in the aqueous stream 250 may be less than about 20 wt %.

The aqueous carboxylic acid stream 250 is supplied to the reactive distillation esterification column 220 together with an alcohol stream 255, though they commonly enter through different inlets in the column 220. Within the reactive distillation esterification column 220, the carboxylic acid reacts with the alcohol to form a corresponding ester. The reactive distillation column simultaneously promotes the esterification reaction and separates the ester from the reaction mixture by vapor-liquid separation. In specific embodiments, the ester can be removed from the column as a top product ester-containing stream 260 as the ester is formed.

In addition to producing the ester-containing stream 260, the reactive distillation esterification column 220 can separate non-volatile components of the aqueous feed stream. For example, water, dissolved salts, electrolytes, or other impurities can remain in a bottoms stream 270 discharged from the column. In the example illustrated in FIG. 2, part or all of the bottoms stream 270 can be recycled to the carbon oxide electrolyzer system 210. In specific embodiments, a separator (not shown), for example a reverse osmosis filter, can split a portion (e.g., stream 272) from the bottoms stream 270. In this example, stream 272 can comprise water to act as a reactant to the hydrolysis of the ester. The remainder of stream 270 can contain electrolytes that can be used in the electrolyzer system 210.

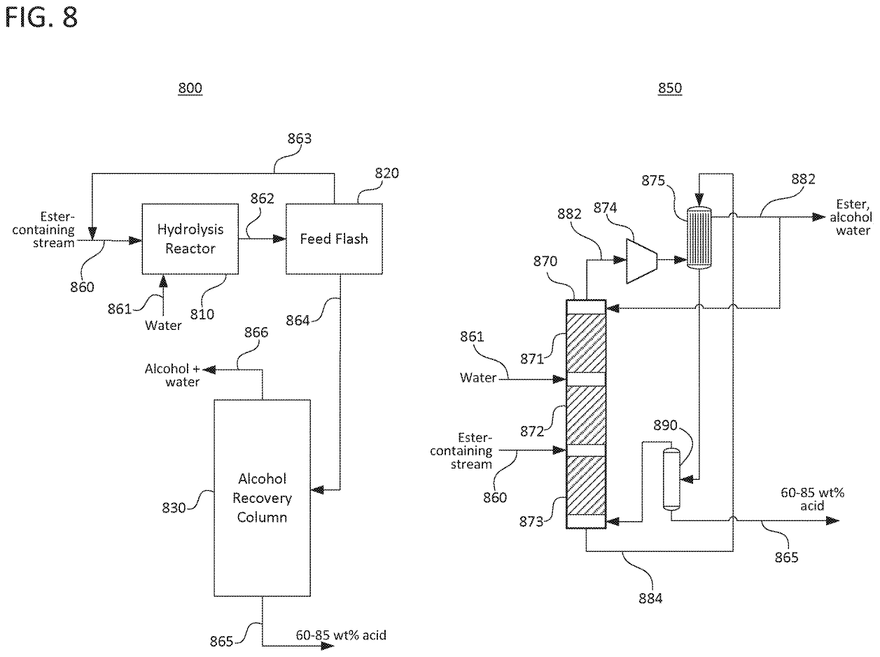

The ester-containing stream 260 can be supplied to the hydrolysis and separation system 230. A hydrolysis subsystem in the system 230 converts at least a portion of the ester to the original carboxylic acid and alcohol. A purified and concentrated carboxylic acid stream 265 can be output directly as a product. In specific embodiments, acid stream 265 can be further purified in an optional drying unit 245, which can output a substantially anhydrous acid product 267 and can also act as a separate source of water for the hydrolysis and separation system 230 (i.e., water from the drying unit 245 can be combined with water stream 272 and used in the hydrolysis reaction). In specific embodiments, the hydrolysis and separation system 230 can comprise a hydrolysis reactor in which the ester reacts with water to regenerate the carboxylic acid and alcohol, followed by separation of the alcohol from the regenerated acid. Alternately, the hydrolysis and separation system may comprise a hydrolysis reactive distillation column in which hydrolysis and separation occur within the same unit. Various hydrolysis configurations will be discussed in more detail with reference to FIG. 8. In specific embodiments, the hydrolysis and separation system 230 can include one or more separation units before and/or after the hydrolysis subsystem to separate out various water, alcohol, acid, or ester portions of internal streams, including an ester/alcohol separator unit or an acid/alcohol separator unit. Output stream 280 can consist largely of alcohol that is a product of hydrolysis, though this stream can also contain residual alcohol that was present in the ester-containing stream 260. In this configuration, feed stream 259 may be omitted, or could be present to input make-up alcohol to account for internal losses. Since the focus of this embodiment is outputting a purified carboxylic acid product, alcohol is substantially used as an intermediate that is reacted in the esterification column 220 and regenerated in the hydrolysis and separation system 230. Depending on design and reaction constraints, in specific embodiments, the alcohol input stream 255 can enter the column in the middle near the reaction zone. In other variants, the alcohol input stream can enter the column at stream 257, where alcohol can be allowed to flow down the column as a reflux.

In specific embodiments, the hydrolysis and separation system can be operated to produce a carboxylic acid product stream having a concentration greater than that of the incoming aqueous feed stream. For example, when processing dilute aqueous streams containing less than about 20 wt % carboxylic acid, the system can produce a recovered carboxylic acid stream having a concentration greater than about 50 wt %, and in some embodiments about 60-85 wt %, prior to any optional downstream purification steps, such as an acid drying step.

The configuration illustrated in FIG. 2 can support multiple operating regimes of the reactive distillation esterification column 220. In specific embodiments, the amount of carboxylic acid and alcohol supplied to the esterification column 220 can be substantially the same. In specific embodiments, the reactive distillation column may operate in an acid-excess mode in which the carboxylic acid is present in molar excess relative to the alcohol and conversion of the acid to ester is intentionally limited per pass, for example <90%. Operation in an acid-excess regime can be advantageous for specific carboxylic acids having two or more carbon atoms, such as acetic acid or propionic acid, where maintaining a portion of unreacted acid in the column can help suppress formation of multi-component azeotropes, which simplifies separation of the ester from water and residual acid at the top output of the column 220. Because alcohol in this configuration is substantially reacted in the reactive distillation esterification column 220, the overhead ester-containing stream 260 may have only small amounts of alcohol. After hydrolysis in the hydrolysis and separation system 230, there will be both concentrated carboxylic acid as well as alcohol present in the stream. These can be separated in an acid/alcohol separation subsystem.

In specific embodiments, the reactive distillation column may operate in an alcohol-excess mode in which alcohol is supplied in molar excess relative to the carboxylic acid. Alcohol-excess operation can be advantageous for specific lower molecular weight carboxylic acids, such as formic acid, where high conversion of the acid to ester per pass can promote efficient removal of the ester from the column. In such embodiments, the ester-containing stream 260 may include both ester and alcohol, and the hydrolysis and separation system 230 can be configured differently than when using an acid excess operating regime. For example, if there is a substantial amount of alcohol mixed in with the ester-containing stream 260, an ester/alcohol separation system can be used before further processing. The ester/alcohol separation system can be placed upstream of the hydrolysis portion. Selection of the operating regime can therefore depend on the molecular structure of the carboxylic acid, the volatility relationships among the alcohol, ester, water, and acid components, and the desired configuration of downstream processing units.

In specific embodiments, systems such as system 200 can incorporate thermal integration between process units to improve overall energy efficiency. For example, heat generated in upstream processes, including carbon oxide electrolyzer systems or exothermic reaction steps, can be used to supply thermal energy to one or more downstream units, such as reboilers associated with reactive distillation esterification columns or separation columns. In specific embodiments, heat pump systems or mechanical vapor recompression systems can be used to recover heat from process streams and redistribute thermal energy within the system. Such thermal integration can reduce overall external heating requirements and improve process efficiency.

The systems described herein are particularly advantageous for processing dilute aqueous carboxylic acid streams in which the carboxylic acid concentration is relatively low. In specific embodiments, the aqueous feed stream supplied to the reactive distillation esterification column can contain less than about 20 wt % carboxylic acid. At such dilute concentrations, conventional purification techniques based on direct distillation of the acid from water can become inefficient due to the similar boiling points and potential azeotropic behavior of the components. Converting the carboxylic acid to a corresponding ester within the reactive distillation column can therefore provide a mechanism for selectively removing the acid from the dilute aqueous stream in a form that is more readily separable from water and other components.

While the embodiment illustrated in FIG. 2 focuses on regeneration of the original carboxylic acid through hydrolysis of the ester intermediate, the systems described herein can alternatively be configured to recover the ester as a final product. In some industrial environments, it can be advantageous to valorize the ester rather than regenerating the carboxylic acid. In some cases, the economic value of the ester can exceed the value of both the original carboxylic and the alcohol used to create it. For example, facilities that produce or process alcohols together with carbon oxide streams, such as ethanol production plants with carbon dioxide capture systems, petrochemical complexes, or integrated biorefineries, can have ready access to both alcohol and carbon oxide feedstocks. In addition, ester products can function as transportable intermediates for downstream chemical conversion. Esters such as methyl formate can act as carriers for carboxylic acids, carbon monoxide, or hydrogen and can be converted at other facilities to produce these chemicals or other related products. As a result, ester production can allow distributed production sites to convert dilute carboxylic acid streams into stable liquid intermediates that can be transported to centralized processing facilities.

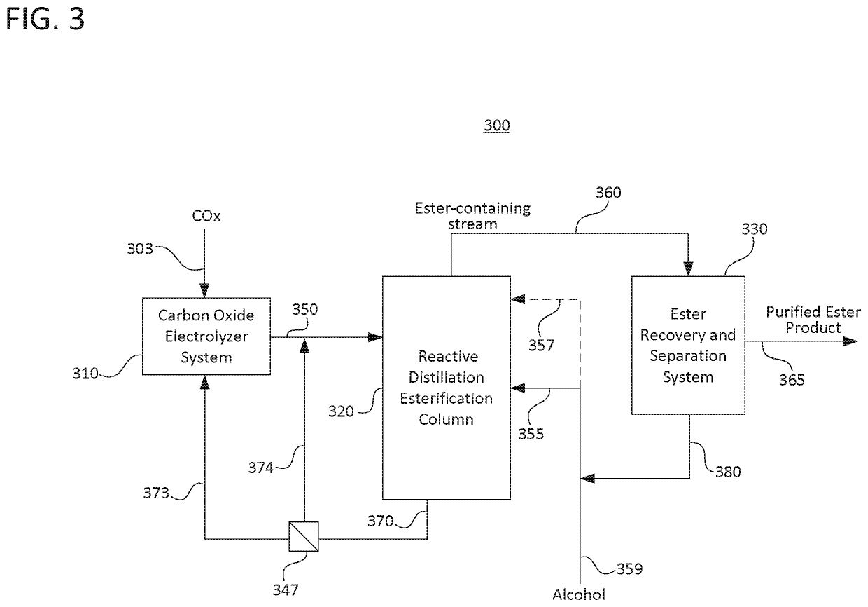

One example of such a system configured to recover a purified ester product is illustrated in FIG. 3, which shows a diagram of a system 300 for producing a purified ester product in accordance with specific embodiments of the inventions disclosed herein. System 300 includes a carbon oxide electrolyzer system 310. Though an electrolyzer is shown in this example, any of the other dilute carboxylic acids discussed in regard to system 100 of FIG. 1 could be used. In this example, the carbon oxide electrolyzer system 310 produces a dilute aqueous carboxylic acid stream 350 from a carbon oxide source 303. The acid stream 350 is supplied to a reactive distillation esterification column 320.

The aqueous carboxylic acid stream 350 is supplied to the reactive distillation esterification column 320 together with an alcohol stream 355, though the aqueous stream 350 and alcohol stream 355 can enter the column through different inlets. Within the reactive distillation esterification column 320, the carboxylic acid reacts with the alcohol to form a corresponding ester. The reactive distillation column simultaneously promotes the esterification reaction and separates the ester from the reaction mixture by vapor-liquid separation. In specific embodiments, the ester can be removed from the column as a top product ester-containing stream 360 as the ester is formed. The production of the ester-containing stream 360 is quite similar up to this point as in system 200 of FIG. 2 which produces an ester containing stream 260.

In addition to producing the ester-containing stream 360, the reactive distillation esterification column 320 can separate non-volatile components of the aqueous feed stream. For example, water, dissolved salts, electrolytes, or other impurities can remain in a bottoms stream 370 discharged from the column. In the example illustrated in FIG. 3, part or all of the bottoms stream 370 can be recycled to the carbon oxide electrolyzer system 310. In specific embodiments, a separator (not shown), for example a reverse osmosisfilter, a nanofiltration separator, or other membrane separation unit, can receive at least a portion of the bottoms stream 370 and produce a separated stream. In such embodiments, the separated stream can comprise water suitable for reuse within the process, for example within the electrolyzer system 310 or other portions of the system. In this embodiment, there is no need for a separate source of water for hydrolysis. In specific embodiments, there can be an optional membrane separator 347 that routes a water and electrolyte stream 373 back to the electrolyzer system 310, and an unreacted carboxylic acid stream 374 to the reactive distillation esterification column 320.

The ester-containing stream 360 can be supplied to an ester recovery and separation system 330. System 330 can separate the ester from alcohol, water, or other components present in the ester-containing stream 360 to produce a purified ester product stream 365. In specific embodiments, the ester purification system 330 can comprise one or more distillation columns, membrane separation systems, decantation systems, or combinations thereof configured to separate the ester from other components of the stream.

In specific embodiments, the ester purification system 330 can produce an alcohol-containing recycle stream 380 that is returned to the reactive distillation esterification column 320. In such embodiments, alcohol recovered in the purification system can be reused as a reactant in the esterification reaction. In this configuration, the alcohol functions primarily as a reactant as any recirculated alcohol was unreacted. In this case, a feed alcohol stream 359 can be supplied as alcohol is consumed in the ester production process.

Similarly to system 200 in FIG. 2, in specific embodiments, the reactive distillation esterification column 320 can receive alcohol through one or more inlets. For example, the alcohol stream 355 can be introduced near a reaction zone of the column where esterification occurs. In other embodiments, an alcohol stream 357 can be introduced at an upper portion of the column where the alcohol can flow downward through the column as reflux. The selection of alcohol feed locations can depend on the desired reaction conditions and separation behavior within the column.

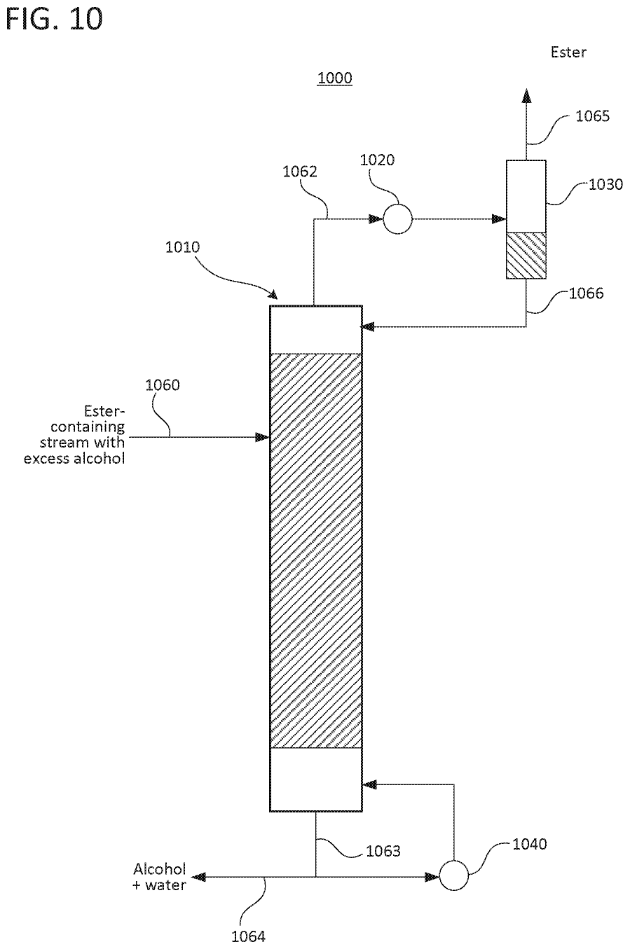

The configuration illustrated in FIG. 3 can support multiple operating regimes of the reactive distillation esterification column 320 similar to those described with respect to FIG. 2. Discussion of the acid excess or the alcohol excess regime is substantially the same as was previously discussed in reference to FIG. 2 as the reaction conditions are substantially the same in the reactive distillation esterification column up to the production of the ester-containing stream 360. In system 300, when running in an acid-excess regime, optional membranes can be used to separate carboxylic acid from water. Membranes can also be used to separate out the ester with >99 wt % purity. When running in an alcohol-excess regime, the ester recovery and separation system 330 can further comprise an ester/alcohol separation system, which will be described later in FIG. 10. The selection of which regime to use can depend on a variety of factors. For example, carboxylic acids having two or more carbon atoms (C2+) often favor operation in the acid excess regime, whereas carboxylic acids having a single carbon atom (C1) such as formic acid may be more suitably processed in an alcohol excess regime.

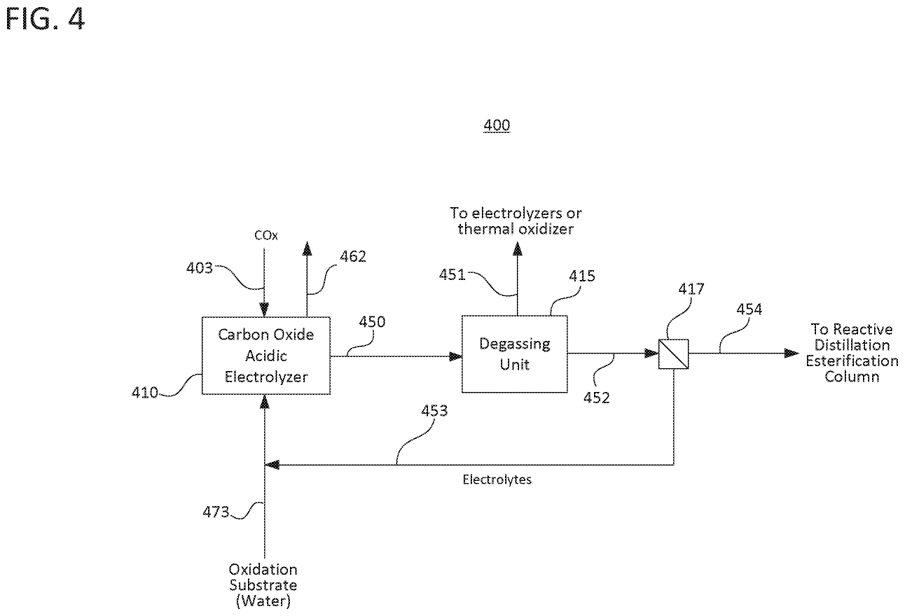

FIGS. 4-11 illustrate detailed examples of subsystem components that can be incorporated into the platform architectures described with respect to FIGS. 1-3. The subsystems illustrated in these figures represent example implementations of process units that can be used to generate dilute carboxylic acid streams, carry out esterification reactions, perform hydrolysis and separation of ester intermediates, recover alcohol from various compositions for recycle, separate ester products, or further purify carboxylic acid products. In specific embodiments, one or more of the subsystems described with respect to FIGS. 4-11 can be combined within a single system such as system 100, system 200, or system 300 described previously. In other embodiments, only a subset of the illustrated subsystems may be present depending on the desired configuration of the process and the chemical products to be produced.

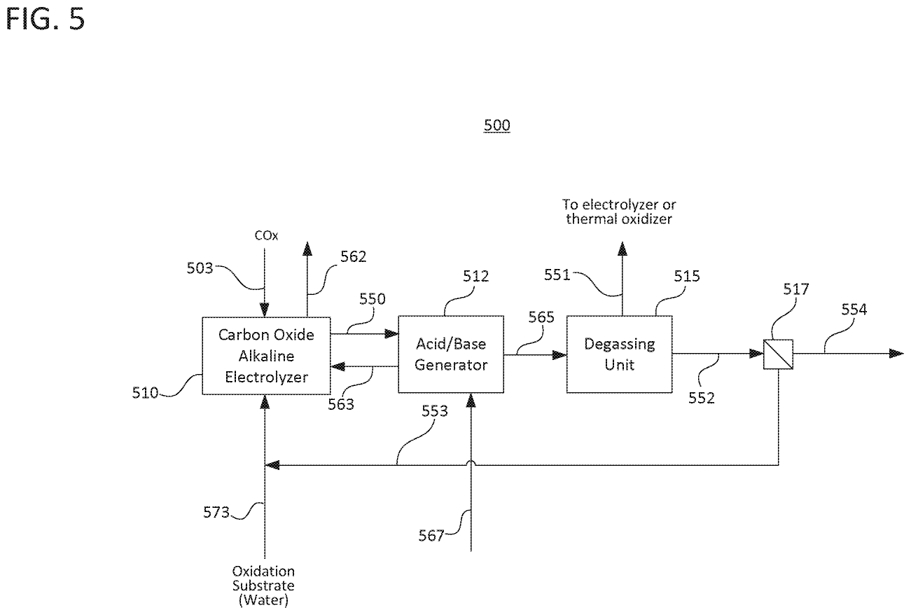

The dilute aqueous carboxylic acid streams supplied to the system described with respect to FIG. 1 can originate from carbon oxide conversion processes. Carbon oxide electrolyzer systems were shown in FIGS. 2 and 3 that can convert CO, CO₂, or mixtures thereof including streams generated from upstream carbon oxide electrochemical conversion processes. In specific embodiments, the carbon monoxide supplied to the electrolyzer may be produced by an upstream CO₂ electrolyzer configured to convert CO₂ to CO. Though gaseous products (for example, hydrogen or ethylene) can be produced in such systems at the same time as liquid products, electrolyzers can be configured to efficiently produce carboxylic acids or corresponding carboxylate species. Carbon oxide electrolyzer systems can operate using different electrolyte environments, which affects the form in which the carboxylic acid product is generated. For example, some electrolyzer systems operate with acidic electrolytes and can produce carboxylic acids directly in the electrolyte stream, while other systems operate with alkaline electrolytes and can produce carboxylate salts. These can be subsequently converted to the corresponding carboxylic acids using an acid/base generator such as an electrodialysis bipolar membrane (EDBM) unit or a salt-splitting unit. Examples of electrolyzer systems that can supply dilute carboxylic acid streams to the reactive distillation esterification systems described herein are illustrated in FIGS. 4 and 5.

### Acidic Electrolyzer System

FIG. 4 shows a diagram of an acidic electrolyzer system 400 in accordance with specific embodiments of the inventions disclosed herein. The acidic electrolyzer system 400 represents one example of a dilute carboxylic acid source that can supply an aqueous carboxylic acid stream 250 or 350 to the systems described with respect to FIGS. 2 and 3 in electrolyzer systems 210 and 310 respectively.

The acidic electrolyzer system 400 includes a carbon oxide acidic electrolyzer 410 configured to convert carbon monoxide, carbon dioxide, or mixtures thereof into one or more carboxylic acids. The electrolyzer 410 receives a carbon oxide feed stream 403 and an oxidation feed stream 473. The oxidation feed stream can comprise water or another oxidizable substrate supplied to the anode of the electrolyzer and may be supplied externally from the overall system or recycled from other components. Within the carbon oxide electrolyzer 410, electrochemical reactions convert the carbon oxide feed into one or more products including a carboxylic acid. In this example, the electrolyzer operates with an acidic electrolyte that allows the carboxylic acid to be produced directly in the electrolyte stream. As a result, an electrolyte stream 450 containing a dilute carboxylic acid can be withdrawn from the electrolyzer and sent to a downstream esterification system or to one or more optional separation steps before esterification. In specific embodiments, the concentration of carboxylic acid in stream 450 can be less than about 20 wt %. The electrolyzer can also generate one or more anode off-gas streams 462, which can comprise oxygen or other gaseous products generated during operation of the electrolyzer.

In specific embodiments, the electrolyte stream 450 can be supplied to a degassing unit 415. The degassing unit 415 removes dissolved gases such as oxygen, hydrogen, carbon monoxide, carbon dioxide, nitrogen, argon, ethylene, or other gaseous species that may be present in the electrolyte stream 450. The degassing unit 415 can work using a combination of low pressure and heating. The water-based electrolyte is heated to near its bubble point (e.g., 90-105° C.), which causes the gases to vaporize and separate from the liquid. Removal of such gases into a degas stream 451 can improve the stability of downstream separation processes. Output stream 452 can be directly sent to a downstream esterification system or passed to another separator. The degas stream 451 can be sent to a thermal oxidizer, or portions can be recycled back to an electrolyzer for reuse, such as electrolyzer 410. In specific embodiments, the degasser can use an atmospheric stripping column with a reboiler and feed product exchanger to remove dissolved gases.

In specific embodiments, the acidic electrolyzer system 400 can optionally include one or more purification units 417 upstream of the reactive distillation system. For example, purification unit 417 can comprise a reverse osmosis unit, a filtration system, an ion exchange unit (in specific embodiments implemented with an ion exchange resin bed), or other separation equipment configured to remove salts or other impurities from the electrolyte stream prior to introduction into the reactive distillation esterification column. A portion of the electrolyte can be recycled through an electrolyte recycle stream 453 to the carbon oxide electrolyzer 410 in order to maintain electrolyte composition and conductivity within the electrolyzer system. The electrolyte recycle stream 453 can be added to the oxidation feed stream 473 or can be added in a separate inlet.

Following these one or more optional separation steps, the electrolyte stream forms a dilute aqueous carboxylic acid stream 454, which can be supplied as the aqueous carboxylic acid feed stream to the reactive distillation esterification systems described with respect to FIGS. 2 and 3.

### Alkaline Electrolyzer System

FIG. 5 shows a diagram of an alkaline electrolyzer system 500 in accordance with specific embodiments of the inventions disclosed herein. Similar to the acidic electrolyzer system described with respect to FIG. 4, the alkaline electrolyzer system 500 represents another example of a dilute carboxylic acid source that can supply an aqueous carboxylic acid stream to the systems described with respect to FIGS. 2 and 3.

The alkaline electrolyzer system 500 includes a carbon oxide alkaline electrolyzer 510 configured to convert carbon monoxide, carbon dioxide, or mixtures thereof into one or more carboxylate species. The electrolyzer receives a carbon oxide feed stream 503 and an oxidation feed stream 573 (e.g., water). In this embodiment, the electrolyzer operates with an alkaline electrolyte. As a result, the electrochemical reactions occurring within the electrolyzer produce carboxylate salts rather than the corresponding carboxylic acids.

An electrolyte stream 550 containing the carboxylate species, and possibly some hydroxide, can be withdrawn from the electrolyzer and supplied to an acid/base generator 512. In specific embodiments, the acid/base generator 512 can comprise an EDBM unit or a salt-splitting electrolyzer configured to convert the carboxylate species into the corresponding carboxylic acid.

Within the acid/base generator 512, the carboxylate-containing electrolyte stream 550 is separated into two product streams. A carboxylic acid stream 565 is produced that contains the corresponding carboxylic acid in aqueous solution. Stream 565 can be supplied as the dilute aqueous carboxylic acid feed stream to the reactive distillation esterification systems described with respect to FIGS. 2 and 3. The acid/base generator 512 can also produce a regenerated electrolyte stream 563, which can contain hydroxide or other electrolyte species. In specific embodiments, stream 563 can be recycled to the carbon oxide electrolyzer 510 to maintain the electrolyte composition within the electrolyzer system. A makeup water stream 567 can be added to the acid/base generator 512 from an external source, or this may be supplied as separated from the bottoms liquid of a downstream reactive distillation esterification column.

The alkaline electrolyzer system 500 can optionally include one or more purification units such as a degassing unit 515 or a purification unit 517 upstream of the reactive distillation system. A stream 552 may connect the output of the degassing unit 515 to the input of the purification unit 517. These are configured like similar systems as described with respect to system 400 in FIG. 4 but may generate a separated gas stream 551, a recycled electrolyte stream 553, or an output stream 554. The electrolyzer system can also generate an anode off-gas stream 562 that contains gaseous products such as oxygen.

In specific embodiments, an alkaline electrolyzer 510 can be configured to convert carboxylate salts produced in the cathode compartment into a dilute carboxylic acid stream in its anode compartment, removing the need for a separate acid/base generator.

### CO₂ Electrolyzer

In specific embodiments, the carbon oxide electrolyzer 410 or 510 with respect to FIGS. 4 and 5 comprises a CO₂ electrolyzer. A CO₂-rich feed gas is routed to the CO₂ electrolyzer. The CO₂ electrolyzer is comprised of a cathode that undertakes the transformation of CO₂ into a carboxylic acid and/or a carboxylate. The cathode catalyst may contain one or more catalytic metals selected from Ag, Au, Cu, Sn, Pb, Bi, Zn, Cd, Ni, Fe, Co, Mn, Mo, In, Pd, Pt, Ti, W, Sb, V, Cr, Rh, or Re. In specific embodiments, the cathode may also generate hydrogen or carbon monoxide as byproducts.

The electrolyzer also comprises an anode that undertakes oxidation of a substrate. Examples of oxidation substrates include water, hydrogen, biomass, organic compounds, halides, or other oxidizable species. The anode typically contains a catalyst comprised of metals such as Ir, Ru, Ni, Co, Fe, Mn, Cu, Pt, Ti, Sn, Mo, W, Cr, V, or Pb.

In specific embodiments, the electrolyzer includes one or more separators positioned between the anode and cathode. A separator may be an anion exchange membrane, a cation exchange membrane, a neutral separator, a bipolar membrane, or combinations thereof.

In specific embodiments, the electrolyzer includes a compartment dedicated to collection of carboxylic acid and/or carboxylate through a separate stream. This stream may include materials or species intended to increase conductivity, such as ion exchange resins or inert electrolytes. In specific embodiments, acidic equivalents produced at the anode can be used to acidify a carboxylate produced at the cathode.

In specific embodiments, the electrolyzer includes a falling film of electrolyte flowing adjacent to the cathode to promote electronic selectivity and collect the carboxylate or carboxylic acid produced from CO₂ conversion. In specific embodiments, the falling film can be promoted using a percolator layer.

In specific embodiments, a CO₂ electrolyzer can be run using an acidic or an alkaline electrolyte. The most common outputs from a CO₂ electrolyzer are C1 products such as formic acid or formate when running in an acidic or alkaline mode respectively. Though they are capable of producing C2 or higher carboxylic acids with specific catalysts, these often have low specificity of product.

### CO Electrolyzer

In specific embodiments, the carbon oxide electrolyzer 410 or 510 with respect to FIGS. 4 and 5 comprises a CO electrolyzer. A CO-rich feed gas is supplied to the CO electrolyzer system. In addition to generating gaseous products, the CO electrolyzer system generates a dilute electrolyte stream containing carboxylic acids or carboxylates. Details of the structure, including cathode, anode, and separators, of a CO electrolyzer are similar to the CO₂ electrolyzer described earlier.

A CO electrolyzer is a device comprising of a cathode area where CO reduction takes place, according to Eq. 2 below, and an anode area where an oxidation reaction takes place on an oxidizing catalyst. The oxidation substrate can be hydroxide, water, dihydrogen gas, halides, organic waste, or any other oxidation substrate. For example, the oxidation can involve water oxidation or dihydrogen oxidation according to Eqs. 3 and 4 below respectively.

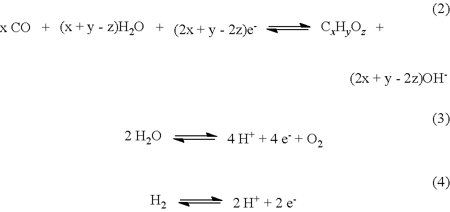

The reactions below can be conducted in accordance with the electrolyzer assemblies described herein. In the diagrams provided herein, only single cells are represented for clarity, but these could be assembled in a plurality of cells, such as in an electrolyzer stack. A CO electrolyzer comprises a cathode comprising a gas-diffusion layer and a cathode catalyst, and the anode comprises an anode catalyst deposited on a transport layer of any shape (such as but not limited to a foam, a mesh, a deposit onto a conductive porous transport layer (PTL), etc.). In this case, the carbon monoxide reduction products include one or more of the following: ethylene (C₂H₄), acetic acid (CH₃COOH), and propionic acid (C₂H₅COOH). CO reduction reactions in neutral/alkaline conditions include:

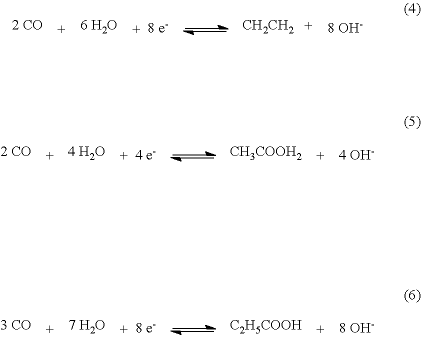

Carboxylates are generated directly at the cathode from CO reduction. In specific embodiments, the generated negatively charged carboxylates can travel to the positively charged anode due to electromigration. RCOOM are then formed as described below, M being alkali metal (Na⁺, K⁺, Li⁺, or Cs⁺) and R being acetate, propionate, or other carboxylate anions.

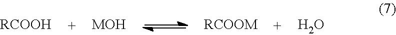

In specific embodiments, the CO electrolyzer can be configured to convert CO to multi-carbon products. CO electrolysis systems can operate with acidic electrolytes, but this is a less common approach. They typically operate in neutral or alkaline electrolytes and can produce C₂ or higher carbon products through carbon-carbon coupling reactions occurring at the cathode catalyst. In specific embodiments, the CO electrolyzer produces carboxylates such as acetate or propionate that can subsequently be converted to the corresponding carboxylic acids as described previously with respect to FIG. 5. CO electrolyzers operating with neutral electrolytes typically convert CO to carboxylates at the cathode under locally alkaline conditions. Carboxylates thus produced in the neutral electrolyte CO electrolyzer can be treated similarly to those produced in alkaline electrolytes and thus processed the same as described with respect to system 500 in FIG. 5.

### Reactive Distillation Esterification Column

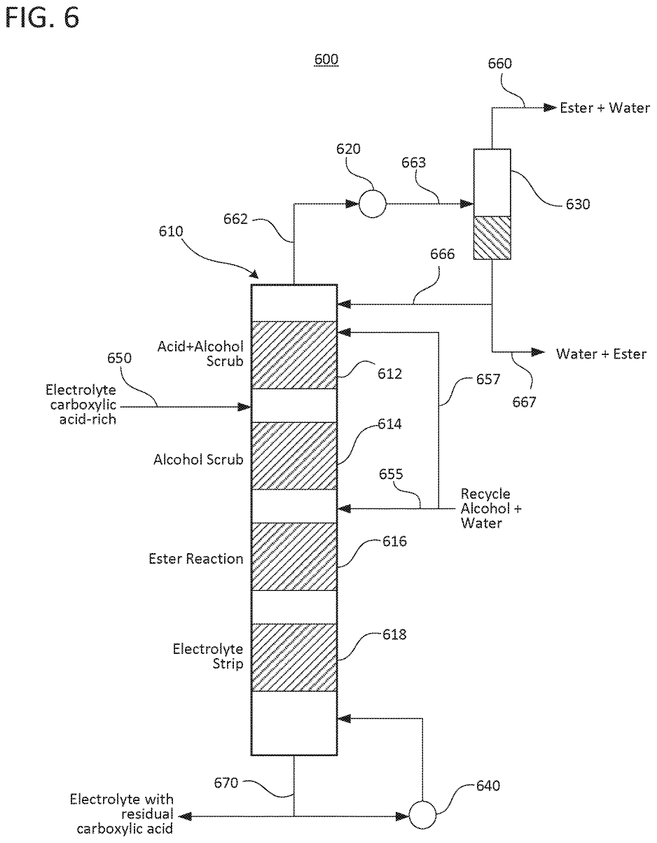

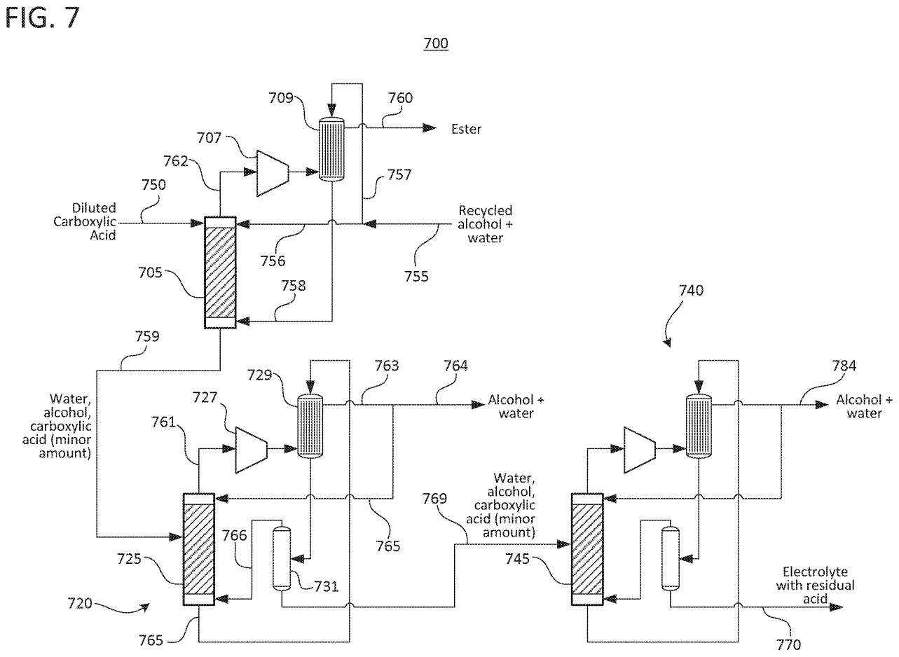

The reactive distillation esterification columns illustrated schematically in FIGS. 1, 2, and 3 can be implemented using a variety of column configurations that promote both esterification reactions and vapor-liquid separation within the same process unit. In specific embodiments, the reactive distillation esterification column can include one or more reaction zones and one or more vapor-liquid contacting stages configured to promote esterification of a carboxylic acid with an alcohol while simultaneously separating an ester product from the reaction mixture. Example configurations of reactive distillation esterification systems that can be used in the processes described herein are illustrated in FIGS. 6 and 7, although other configurations are possible and might vary depending on the acid and the alcohol chosen.

FIG. 6 shows a diagram of a single-column reactive distillation esterification system 600 in accordance with specific embodiments of the inventions disclosed herein. The reactive distillation esterification system 600 includes a reactive distillation column 610 that integrates reaction and separation within a single column vessel. In the embodiment illustrated in FIG. 6, the reactive distillation column 610 includes multiple functional zones that promote different stages of the esterification and separation processes.

In specific embodiments, the reactive distillation column 610 includes an acid/alcohol scrubbing section 612, an alcohol scrubbing section 614, an ester reaction section 616, and a lower electrolyte stripping section 618. In specific embodiments, the reaction section 616 can include structured catalytic packing, catalyst-coated packing materials, catalyst-containing trays, or other internals configured to promote esterification reactions within the distillation column, where the catalysts are capable of promoting esterification of the carboxylic acid with the alcohol under acidic conditions within the reactive distillation column.

In operation, an aqueous carboxylic acid-rich feed stream 650 and an alcohol feed stream 655 are introduced into the reactive distillation column 610. In specific embodiments, the aqueous carboxylic acid feed stream 650 can be introduced into the scrubbing section 612 while the alcohol feed stream 655 can be introduced into or near the reaction section 616. In other embodiments, one or more additional alcohol feed streams such as stream 657 can be introduced at different locations within the column to control reaction conditions and internal reflux behavior.

Within the reaction section 616, the carboxylic acid reacts with alcohol to form an ester and water. Because the ester product is typically more volatile than the carboxylic acid under the operating conditions of the column, the ester can vaporize and rise toward the top of the column while heavier components remain in the liquid phase and flow downward.

An ester-containing vapor stream can be withdrawn from the top of the column and supplied to a condenser 620. The condenser 620 condenses the vapor stream and supplies the condensed liquid 663 to a separator 630. In specific embodiments, the separator 630 can be a reflux drum or phase separator as a decanter as shown in FIG. 6. In specific embodiments, the separation can be done using membrane separation units including, but not limited to, reverse osmosis and/or membrane distillation for selective separation of ethanol from water, ester from water, or carboxylic acid from water. In specific embodiments, separation can be done by one or more distillation columns. In specific embodiments, combinations of the above techniques can be used. This separation step generates at least two streams: a recycle stream containing water and carboxylic acid returned to the esterification section, and a concentrated ester and water stream directed to the hydrolysis system. Additionally, it may also produce an alcohol-rich stream to be recycled to the esterification reactive distillation system. At least a portion of the aqueous phase can be returned to the column as an aqueous reflux stream 666, which can enter the scrubbing section 612 to remove trace amounts of carboxylic acid or other components from rising vapors. Another portion 667 of the aqueous phase can be pumped to a downstream reactor (for example, a hydrolysis reactor, if present in the system). The ester-rich phase can be withdrawn as an ester phase stream 660 and supplied to downstream processing systems such as those described with respect to FIGS. 2 and 3. Compositions of stream 660 and portion 667 will be as stated when the column 610 is run using the regime of an excess of acid as input. When running in the regime of an excess of alcohol, there will be excess alcohol in the vapor stream 662, and other separation methods may be needed. Note that in reference to FIG. 2, this first separator 630 could be shown as part of the hydrolysis and separation system 230, but is included here for clarity, as the separation is tightly integrated with the process flow of the esterification column.

Liquid flowing downward through the column can enter the electrolyte stripping section 618, where heat supplied by a reboiler 640 promotes removal of residual alcohol and ester from the liquid phase. These lighter components can vaporize and return to the reaction section, while a heavier bottoms stream 670 containing water, residual carboxylic acid, salts, or other non-volatile impurities can be withdrawn from the bottom of the column. In specific embodiments, part or all of the bottoms stream 670 can be recycled to an upstream process such as a carbon oxide electrolyzer system or other dilute carboxylic acid source. A portion of the bottoms stream can be routed to the reboiler 640 as well.

FIG. 7 shows a diagram of a multi-section reactive distillation esterification system 700 in accordance with specific embodiments of the inventions disclosed herein. In contrast to the single-column configuration shown in FIG. 6, the system 700 distributes the reaction and separation functions across multiple interconnected units. Such configurations can provide additional flexibility in controlling reaction conditions, pressure profiles, and separation performance.

In the embodiment illustrated in FIG. 7, the esterification system 700 comprises a reaction zone or column and one or more stripping zones or columns. In specific embodiments, the reaction zone and the stripping zones can each be operated at a different pressure. This enables the reaction zones and stripping zones to function as independent distillation columns. The example shown in FIG. 7 includes a reaction column 705 in which esterification of the carboxylic acid with the alcohol occurs, and two stripping columns 725 and 745. Each of the columns includes a mechanical vapor recompression (MVR) section, which can recover heat otherwise lost in the condenser section at the top of the column and use it for the reboiler section at the bottom. For example, the MVR section for the reaction column 705 comprises a compressor 707 and a heat exchanger 709. The use of two stripping zones in this example, rather than a single zone, reduces the overall temperature gradient within the column and thereby improves the thermodynamic efficiency of the MVR process, but a single stripping zone could be used alternatively.

An aqueous carboxylic acid feed stream 750 and an alcohol feed stream 755 can be introduced into the reaction column 705 to promote formation of the ester. The reaction column 705 can contain a catalytic portion similar to that described with respect to the reaction section of FIG. 6. An overhead ester-containing stream 762 can be compressed in a compressor 707 to heat it up and then passed through a heat exchanger 709. The cooled ester-containing stream 760 is output for further downstream processing. Ester-containing stream 760 can also contain water, alcohol, and a minor amount of unreacted carboxylic acid. In specific embodiments, the ester can have a similar separator as described for separator 630 in FIG. 6, along with associated variants.

The alcohol feed stream 755 can be a recycled alcohol stream from other subsystems and can be composed of recycled alcohol from within the esterification system 700. The alcohol stream 755 can be split into two portions: a first portion is introduced at the top of the reaction zone together with the incoming acid feed stream 750 which reacts to form the ester stream 762, while a second portion 757 is preheated by the heat exchanger 709 and sent as stream 758 to the bottom of the column 705. This can allow the second portion 757 to achieve a vapor fraction in the range of 0.8 to 0.99 prior to reintroduction into the system. The alcohol feed stream 755 and the second portion 757 combine to form stream 756. The bottoms stream 759 comprises water, alcohol, and a minor amount of carboxylic acid.

Alcohol can be recovered from the water and carboxylic acids present in the bottoms stream 759 in one or more stripping sections. The first stripping section 720 comprises a stripping column 725, which separates incoming stream 759 into a top stream 761 containing alcohol and water, and a bottoms stream 765 with electrolytes, carboxylic acids, water, and remaining alcohol. The top stream 761 enters an MVR section similar to that of the reaction portion, the MVR section comprising a compressor 727 and a heat exchanger 729. The cooled stream 763 can be mostly output as an alcohol and water stream 764. A portion of stream 763 can be recycled as reflux stream 765 to the stripping column 725. Bottoms liquid stream 765 can be routed through the heat exchanger 729 to pick up heat from stream 761 before entering a flash drum 731. Heated vapor stream 766 can reenter the stripping column 725 at the bottom and act as a reboiler, while a liquid phase enters a second stripping section, in this example.

Similar to the first stripping section 720, the second stripping section 740 comprises a stripping column 745, which separates incoming stream 769 into a top stream containing alcohol and water, and a bottoms stream with electrolytes, carboxylic acids, water, and remaining alcohol. The incoming stream 769 contains water, alcohol, and a minor amount of carboxylic acid output from the flash drum 731. The top stream enters an MVR section similar to that of the reaction portion, the MVR section comprising a compressor and a heat exchanger. The cooled stream can be mostly output as an alcohol and water stream 784. A portion of the cooled stream can be recycled to the stripping column 745. The bottoms liquid can be routed through the heat exchanger to pick up heat from the top stream before entering a flash drum. A heated vapor stream can reenter the stripping column 745 at the bottom and act as a reboiler, while a liquid phase containing electrolytes and residual acid is output as stream 770.

In specific embodiments, the multi-column configuration illustrated in FIG. 7 can allow independent control of reaction conditions, vapor-liquid separation, and stripping performance within separate column units. Such configurations can be advantageous when processing dilute aqueous carboxylic acid streams, where careful control of reaction equilibrium and separation behavior can improve recovery of ester intermediates and overall process efficiency.

Note that reboilers and MVRs were used in the reactive distillation esterification columns shown in FIGS. 6 and 7 respectively, but in specific embodiments, the column designs could be implemented using reboilers, MVRs, or heat pumps alone or in combination.

### Hydrolysis and Separation Systems

The hydrolysis and separation system 230 shown in FIG. 2 can be implemented using a variety of reactor and separation configurations. In specific embodiments, hydrolysis of the ester intermediate can be performed using one or more reactors, reactive distillation columns, or combinations of reaction and separation equipment configured to regenerate the carboxylic acid and alcohol from the ester. FIG. 8 provides diagrams of hydrolysis and separation systems 800 and 850 in accordance with specific embodiments of the inventions disclosed herein and illustrates several example configurations that can be used to implement the hydrolysis and separation system described with respect to FIG. 2.

In one embodiment illustrated in FIG. 8, an ester-containing stream 860 is supplied to a hydrolysis and separation system 800. The ester-containing stream can correspond to the ester-containing vapor stream withdrawn from the reactive distillation esterification column described with respect to FIG. 2 after condensation and any intermediate separation steps. In specific embodiments, a water stream 861 can also be supplied to the hydrolysis system to promote hydrolysis of the ester according to the reverse esterification reaction. For example, when the ester comprises methyl formate, hydrolysis of the ester with water produces formic acid and methanol.

In this example, a hydrolysis and separation system 800 includes a hydrolysis reactor 810 configured to convert at least a portion of the ester in stream 860 into the corresponding carboxylic acid and alcohol. The hydrolysis reactor 810 can operate using homogeneous or heterogeneous catalysts suitable for promoting ester hydrolysis, or the reaction can proceed under thermal conditions without an added catalyst depending on process design. In specific embodiments, the reactor 810 can use a strong acid cation resin as a catalyst to hydrolyze most of the ester back to the carboxylic acid and the alcohol. The reaction mixture produced in the hydrolysis reactor can contain the regenerated carboxylic acid, alcohol, water, and residual ester. The excess water content may be controlled so that the product carboxylic acid from the downstream alcohol recovery column meets the required water content. The hydrolysis reactor 810 can be a separate component as shown here but optionally can be included with the downstream alcohol recovery column.

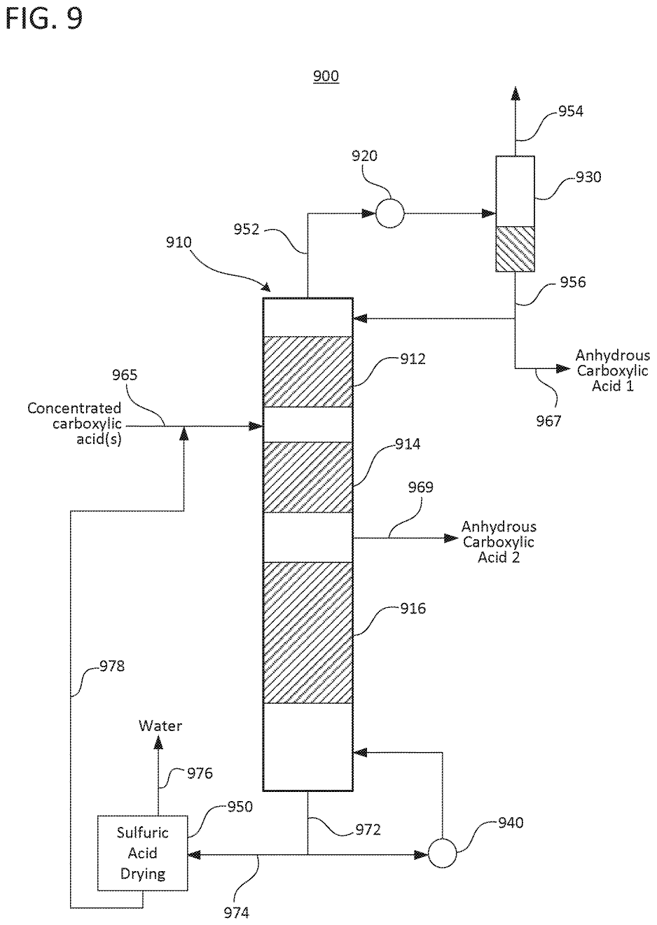

The mixture 862 exiting the hydrolysis reactor 810 can be heated and then supplied to a feed flash drum 820. In these conditions, most of the remaining ester will vaporize and leave as an ester stream 863, that can be recycled back to the input of the hydrolysis reactor 810. The bottoms stream 864 from the feed flash drum 820 can be sent to an alcohol recovery column 830, which is a stripping column where the alcohol is stripped from the carboxylic acid. The alcohol and some of the water exits from the top into stream 866, where it is recycled back to the reactive distillation esterification column. Bottoms liquid stream 865 is a purified, concentrated stream of carboxylic acid, for example at 60-85 wt %. Carboxylic acid stream 865 can be supplied directly as a product or further processed in downstream units such as the optional drying system described with respect to FIG. 9.

In another embodiment illustrated in FIG. 8, hydrolysis and separation system 850 includes a hydrolysis reactive distillation column 870. In this embodiment, the hydrolysis reaction and much of the separation occur in the column 870. An ester-containing stream 860 and a water stream 861 are introduced into the hydrolysis reactive distillation column. The column can include a scrubbing section 871, a reaction section 872, and a stripping section 873. Within the column, hydrolysis of the ester produces the corresponding carboxylic acid and alcohol while vapor-liquid separation simultaneously removes alcohol from the reaction zone. Removal of alcohol from the reaction environment can shift the hydrolysis equilibrium toward formation of the carboxylic acid, increasing overall conversion of the ester.

The hydrolysis reactive distillation column 870 as shown uses an MVR configuration similar to that of stripping section 720 in FIG. 7, though a reboiler and condenser could also be used. The MVR portion can include a condenser 874, a heat exchanger 875, and a flash drum 890. A portion of cooled top output 882 is sent to the top of the column as reflux, while the remainder is recycled back to the first separator (e.g. separator 630 after the esterification column in FIG. 6). In specific embodiments, a second separator can be used to remove alcohol from the recycle stream before it is sent back to the first separator, and the alcohol can be recycled separately to the esterification column. A concentrated carboxylic acid stream 865 (e.g., 60-85 wt %) can be withdrawn from the bottoms stream 884 of the hydrolysis reactive distillation column through flash drum 890.

The configurations illustrated in FIG. 8 represent example implementations of hydrolysis systems that can regenerate a carboxylic acid from an ester intermediate. In embodiments employing a hydrolysis reactor, alcohol produced during hydrolysis can be separated in a downstream alcohol recovery column such as column 830. In embodiments employing a hydrolysis reactive distillation column, alcohol separation can occur within the hydrolysis column itself, thereby eliminating the need for a separate alcohol recovery column. Other reactor and separation configurations can also be used, including combinations of hydrolysis reactors, reactive distillation columns, phase separators, and distillation columns arranged to convert the ester to the corresponding carboxylic acid while recovering alcohol for recycle to the reactive distillation esterification column.

### Acid Drying Unit

In specific embodiments, the carboxylic acid product stream recovered from the hydrolysis and separation system described with respect to FIG. 8 can contain residual water or other volatile components that may be removed to produce a more concentrated or substantially anhydrous carboxylic acid product. FIG. 9 shows a diagram of an acid drying system 900 in accordance with specific embodiments of the inventions disclosed herein. The acid drying system 900 can be used to further purify or concentrate a carboxylic acid product stream produced in the processes described herein.

In the embodiment illustrated in FIG. 9, a carboxylic acid stream 965 is supplied to the acid drying system 900. The carboxylic acid stream 965 can correspond to the concentrated acid stream produced by the hydrolysis and separation system described with respect to FIG. 8 or the acid product stream produced in other embodiments of the processes described herein. In many embodiments, stream 965 can contain a mixture of carboxylic acid and water and may also contain trace amounts of alcohol or other volatile components.

In specific embodiments, the acid drying unit can use distillation, pervaporation, adsorption beds, or other types of acid drying. In the example of FIG. 9, the acid drying system 900 can include a separation column 910 configured to remove water and other volatile components from the carboxylic acid stream. The separation column can operate as a distillation column and can include a condenser 920, a reflux drum 930, and a reboiler 940 to facilitate separation of volatile components from the acid. The column 910 can include multiple separation areas 912, 914, 916 depending on the composition of the carboxylic acid feed stream 965. In operation, the carboxylic acid feed stream 965 is mixed with a concentrated sulfuric acid stream 978. Carboxylic acids and other volatile components can be vaporized within the column and rise toward the top of the column, while water is captured by the sulfuric acid and flows down the column. The top stream 952 can be condensed in the condenser 920 and then passed to a reflux drum 930. Any volatile components remaining are recycled back to upstream sections in stream 954. A portion of the condensed liquid 956 collected in the reflux drum 930 can be returned to the separation column 910 as a reflux stream in order to maintain separation efficiency within the column. The remainder is output as substantially anhydrous carboxylic acid stream 967. The bottoms stream 972 can be partially passed to the reboiler 940, and the remainder of the diluted sulfuric acid stream 974 is passed to a sulfuric acid drying column 950. Water stream 976 removed can be recycled to various other places in the system, for example, to a hydrolysis unit, and the now concentrated sulfuric acid stream 978 is available to be recycled for drying.

In specific embodiments, the concentrated carboxylic acid stream 965 can contain two or more carboxylic acids. A carbon oxide electrolyzer can be tuned to produce more than one carboxylic acid. These will generally flow together through the system until they reach the acid drying system 900. The drying unit provides an opportunity to separate these components while purifying them. The lowest boiling point carboxylic acid will collect from the reflux drum at stream 967. A secondary product carboxylic acid stream 969 with a higher boiling point can be extracted from an intermediate point on the column 910.

### Ester/Alcohol Separation Subsystem

In both systems 200 and 300 in FIGS. 2 and 3, there are places where an ester-containing stream contains alcohol that can be removed. For example, in system 200, in some regimes, the ester stream may have too much alcohol to be efficiently processed. In system 300, where the final product is a purified ester, the final processing step is to remove any remaining alcohol and/or water. An ester/alcohol separation subsystem can achieve both objects. FIG. 10 shows a diagram of an ester/alcohol separation subsystem 1000 in accordance with specific embodiments of the inventions disclosed herein.

In specific embodiments, the reactive distillation esterification column of system 200 with reference to FIG. 2 can be operated in an alcohol-excess regime in which an ester-containing stream withdrawn from the reactive distillation column contains both ester and excess alcohol. The excess alcohol in the distillate stream negatively affects the achievable ester conversion rate in the hydrolysis section, as it shifts the equilibrium reaction back to the ester. It can be less energy intensive to separate the alcohol from the esters upstream of the hydrolysis section than to have a low per pass conversion in the hydrolysis section with increased recycle. In such embodiments, it can be advantageous to remove at least a portion of the alcohol before hydrolysis in order to improve hydrolysis conversion and reduce recycle requirements.

In an embodiment illustrated in FIG. 10, an ester-containing stream 1060 is supplied to the ester/alcohol separation subsystem 1000 into a rectification column 1010. The ester-containing stream 1060 can correspond to an overhead stream withdrawn from a reactive distillation esterification column after condensation and can include ester, alcohol, water, and optionally trace acid. In specific embodiments, the ester-containing stream 1060 can be produced when alcohol is supplied in molar excess relative to the carboxylic acid in the reactive distillation esterification column. In this configuration, the ester-containing stream 1060 may be taken directly from the top of the reactive distillation esterification column or may go through a phase separation first. The ester-containing stream 1060 can be supplied to a separation column 1010 configured to separate at least a portion of the alcohol from the ester-containing stream. In specific embodiments, the separation column can include a condenser and a reflux drum configured to condense overhead vapors and return a portion of the condensed liquid to the column as reflux. The reflux ratio can be adjusted to control the separation between ester and alcohol and to produce an ester product having a desired purity. A portion of the bottoms liquid 1063 containing alcohol and water can be passed to a reboiler 1040, and the rest can exit as stream 1064 to be recycled to the reactive distillation esterification column. The overhead vapor stream 1062 can be condensed in the condenser 1020 and sent to the reflux drum 1030. A portion of the liquid phase 1066 is used as reflux for the column to maintain separation efficiency within the column 1010. Depending on the volatility relationships of the particular ester and alcohol, the separation column 1010 can be operated to produce an ester-rich stream 1065 suitable for downstream hydrolysis and an alcohol-rich stream 1064 suitable for recycle back to the esterification column. When used to remove alcohol before hydrolysis, it is not necessary to remove all of it, is it will undergo downstream processing back to the carboxylic acid. In some cases, some water can remain in the ester stream.

In specific embodiments, subsystem 1000 can be used to purify an ester-containing stream as generated by system 300 in reference to FIG. 3. In this case, the desirable product is a purified product grade ester stream. The structure of the column 1010 is substantially the same as is used within the previous embodiment for separation in preparation for hydrolysis, and so details of the subsystem 1000 will not be discussed again. However, the ester/alcohol separation system can be operated under different rectification conditions depending on the desired function. In embodiments where the column is used upstream of hydrolysis, the column can be operated to remove a majority of the alcohol while allowing a portion of alcohol to remain in an ester-rich stream. In embodiments where the column is used to recover a purified ester product, the column can be operated with increased reflux and separation efficiency to produce an ester stream having a high purity while rejecting alcohol and water to one or more separate streams. The column can be tuned by adjusting the reflux ratio and the energy input to the column. Depending on the relative boiling points of the ester and the alcohol, the output ester stream 1065 can be purified to >99% purity or even higher.

### SYSTEM APPLICATION EXAMPLES

### Example 1—CO₂ Electrolyzer for Formic Acid Production with a Hydrolysis Reactor

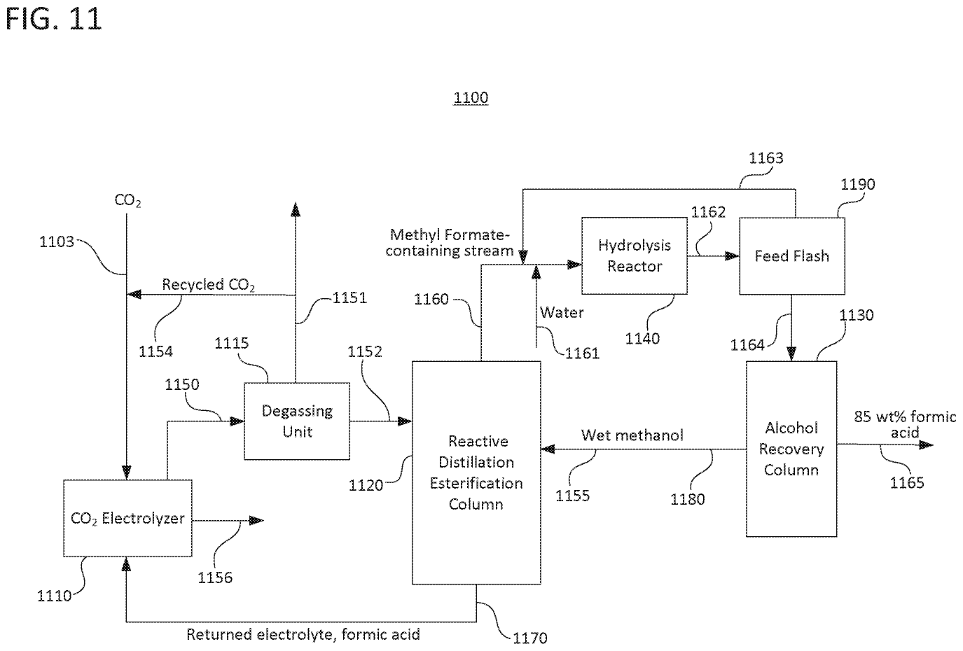

FIG. 11 shows a diagram of a system 1100 for recovering formic acid from a CO₂-derived aqueous stream in accordance with specific embodiments of the inventions disclosed herein. In this example, the system 1100 represents a specific implementation of the acid recovery system 200 described with respect to FIG. 2, in which a CO₂ electrolyzer system, a reactive distillation esterification column, and a hydrolysis and separation system are integrated to recover concentrated formic acid.

In the embodiment illustrated in FIG. 11, a CO₂ electrolyzer system 1110 produces a dilute aqueous stream 1150 comprising formic acid. In specific embodiments, the electrolyzer system 1110 can correspond to the acidic electrolyzer 410 described with respect to FIG. 4. The electrolyte stream 1150 produced by the electrolyzer from a CO₂ source stream 1103 can comprise formic acid, water, dissolved gases including CO₂, and an electrolyte, for example potassium bisulfate or an alkali formate like potassium formate. The electrolyzer system 1110 can also produce a gas product stream 1156, which can contain hydrogen, CO, CO₂, ethylene, or other gases. The electrolyte stream is supplied to a degassing unit 1115 configured to remove dissolved gases. In specific embodiments, the degassing unit operates by heating the stream to near its bubble point, for example about 100-105° C., optionally under reduced pressure, to vaporize dissolved gases such as carbon dioxide. The separated gas stream 1151 can be recycled to the electrolyzer system or partially purged. CO₂ in the gas stream 1151 can be separated into a recycled CO₂ stream 1154 and combined with the CO₂ source stream 1103.

A degassed aqueous stream 1152 comprising formic acid and electrolyte is supplied to a reactive distillation esterification column 1120 together with an alcohol stream 1155 comprising methanol. In specific embodiments, the reactive distillation esterification column 1120 can correspond to the single-column configuration of system 600 described with respect to FIG. 6 and can be operated in an acid-excess regime. Within the column, formic acid reacts with methanol to form methyl formate, which is removed as a methyl formate-containing stream 1160. Methyl formate can be removed readily as it has a low boiling point of 32° C. In specific embodiments, the column is operated such that substantially all of the methanol is converted, while a portion of the formic acid remains unreacted. A bottoms stream 1170 comprising electrolyte and residual formic acid, for example about 10% of the incoming formic acid, is withdrawn and recycled to the electrolyzer system 1110.

In specific embodiments, the ester-containing stream 1160 is supplied to a hydrolysis and separation system, which can correspond to the hydrolysis reactor-based configuration described with respect to system 800 in FIG. 8. The methyl formate-containing stream 1160 is combined with a water stream 1161 and introduced into a hydrolysis reactor 1140 in which methyl formate is hydrolyzed to a formic acid and methanol stream 1162. In specific embodiments, the hydrolysis reactor can include a strong acid ion exchange resin catalyst, for example Amberlyst 15DRY, and can be operated at elevated temperature and pressure, for example about 100° C. and about 2-5 bar(a).

The reaction mixture stream 1162 is supplied to a feed flash vessel 1190 configured to remove residual methyl formate. In specific embodiments, the pressure of the reaction mixture is reduced to promote vaporization of methyl formate, which is recovered as a vapor stream 1163 and recycled to the hydrolysis reactor 1140. A liquid stream 1164 comprising formic acid, methanol, and water is withdrawn from the feed flash vessel and supplied to an alcohol recovery column 1130.

The alcohol recovery column 1130 can separate methanol from the hydrolysis mixture. A methanol-rich overhead stream 1180, which can also contain water, is recovered and recycled to the reactive distillation esterification column 1120 as input methanol stream 1155. A concentrated formic acid stream 1165 is recovered from the bottoms of the column. In specific embodiments, the formic acid product stream 1165 can have a concentration greater than about 50 wt %, for example about 60-85 wt %, and in specific embodiments about 85 wt %., and can be further processed if higher purity is desired. In specific embodiments, heat generated within the system can be integrated between process units as described with respect to system 200.

In specific embodiments, one example of the material balance and process flow of the system in Example 1 is presented in Table 1. Other chemicals or combinations of chemicals (including those not listed or omitting chemicals listed in the table) may be present. Additionally, chemicals may be present in amounts or percentages other than those listed.

TABLE 1
Material Balance of Example 1

| | | | Formic | Methyl | | | | | |
|---|---|---|---|---|---|---|---|---|---|
| | | CO₂ | Acid | Formate | Methanol | KHSO₄ | H₂SO₄ | Water | Total |
| Feed CO₂ | kgmol/h | 45.3 | | | | | | | 45.3 |
| Electrolyzer | kgmol/h | | 5 | | | 21 | 0.4 | 2489 | 2515 |
| Feed | kg/h | 1993 | 232 | | | 2833 | 41 | 43981 | 49079 |
| | wt % | 4.1% | 0.5% | | | 5.8% | 0.1% | 89.6% | 100% |
| Product FA + | kgmol/h | 1 | 50 | | | 21 | 0.4 | 2443 | 2515 |
| Electrolyte | kg/h | 22 | 2315 | | | 2833 | 41 | 43981 | 49192 |
| Degassed FA + | kgmol/h | | 50 | | | 21 | 0.4 | 2489 | 2560 |
| Electrolyte | kg/h | | 2315 | | | 2833 | 41 | 43981 | 49170 |
| | wt % | | 4.7% | | | 5.8% | 0.1% | 89.4% | 100.0% |
| Return | kgmol/h | | 5 | | | 21 | 0.4 | 2489 | 2515 |
| Electrolyte | kg/h | | 232 | | | 2833 | 41 | 44797 | 47902 |
| | wt % | | 0.5% | | | 5.9% | 0.1% | 93.5% | 100.0% |
| Methyl | kgmol/h | | | 45 | | | | 5 | 50 |
| Formate | kg/h | | | 2717 | | | | 91 | 2808 |
| | wt % | | | 96.8% | | | | 3.2% | 100.0% |
| Esterification | kgmol/h | | | | | | | 22.64 | 22.64 |
| Column Int. | kg/h | | 45 | | | | | 408 | 453 |
| Reflux | | | | | | | | | |
| Makeup | kgmol/h | | 2083 | | | | | 66 | 2149 |
| Water | | | | | | | | | |
| Hydrolysis | kgmol/h | | | 76 | | | | 71 | 146 |
| Reactor Feed | kg/h | | | 4529 | | | | 1273 | 5802 |
| Hydrolysis | kgmol/h | | 45 | 30 | 45 | | | | 121 |
| Reactor | kg/h | | 2083 | 1812 | 1449 | | | | 5344 |
| Product | | | | | | | | | |
| Recycle MF | kgmol/h | | | 30 | | | | | 30 |
| | kg/h | | | 1812 | | | | | 1812 |
| Hydrolysis | kgmol/h | | 45 | | 45 | | | | 91 |
| Column Feed | kg/h | | 2083 | | 1449 | | | | 3533 |
| Product FA | kgmol/h | | 45 | | | | | 20 | 66 |
| | kg/h | | 2083 | | | | | 368 | 2451 |
| | wt % | | 85% | | | | | 15% | 100% |
| Wet | kgmol/h | | | | 45 | | | 5 | 50 |
| Methanol | kg/h | | | | 1449 | | | 91 | 1540 |

### Example 2: CO₂ Electrolyzer for Formic Acid Production with a Reactive Distillation Hydrolysis Column

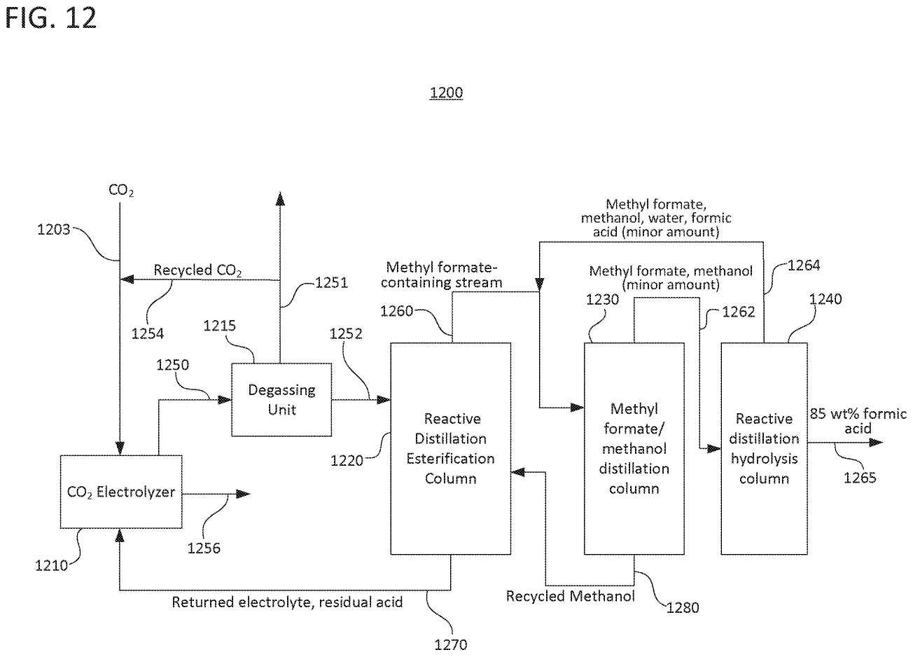

FIG. 12 shows a diagram of a system 1200 for recovering formic acid from a CO₂-derived aqueous stream using reactive distillation esterification and reactive distillation hydrolysis in accordance with specific embodiments of the inventions disclosed herein. The system 1200 represents a specific implementation of the acid recovery system 200 described with respect to FIG. 2, in which a CO₂ electrolyzer system, a reactive distillation esterification system, an intermediate ester/alcohol separation column, and a hydrolysis reactive distillation column are integrated to recover concentrated formic acid.

In the embodiment illustrated in FIG. 12, a CO₂ electrolyzer system 1210 produces a dilute aqueous stream 1250 comprising formic acid. In specific embodiments, the electrolyzer system 1210 can correspond to the acidic electrolyzer 410 described with respect to FIG. 4. The electrolyzer system 1210 and degassing unit 1215 work in the same manner as the electrolyzer and degassing unit as described in system 1100 in FIG. 11. System 1200 also produces an electrolyte stream 1250 from a CO₂ source 1203, a gas product stream 1256, and a separated gas stream 1251 from the degassing unit 1215, part of which can be a recycled CO₂ stream 1254.

The degassed aqueous formic acid stream 1252 with, for example 5 wt % formic acid, is supplied to a reactive distillation esterification system 1220 together with a methanol stream 1280. In specific embodiments, the reactive distillation esterification system 1220 can correspond to the multi-section configuration of system 700 described with respect to FIG. 7 and can be operated in an alcohol-excess regime. In specific embodiments, the esterification system comprises one or more columns that include a central reaction zone and two stripping zones. Within the esterification system 1220, methanol reacts with formic acid in the central reaction zone under acidic conditions within a pressure range, for example, of 0.9 to 3 bara, to produce methyl formate. The stripping zones can operate within a lower pressure range of 0.2 to 0.9 bara. The methyl formate-containing stream 1260 is removed with an overhead vapor stream comprising methyl formate and methanol, possibly along with some water, and a minor fraction of unreacted formic acid. A bottoms stream 1270 comprising electrolyte and a small amount of residual formic acid is withdrawn and recycled to the electrolyzer system 1110. In specific embodiments, methanol supplied to the system can comprise a recycled wet methanol stream 1280 that can be divided into multiple portions and introduced at different locations within the esterification system to control reaction and separation behavior. For example, one portion of methanol can be introduced at the top of the reaction zone together with the incoming acid feed, while a second portion preheated by contact with the column's overhead vapor may be introduced at a different location. This corresponds to the MVR configuration shown in FIG. 7. Stripping zones in the esterification system can recycle methanol to the methanol feed stream 1280.

The overhead vapor from the esterification system is supplied to a methyl formate-methanol separation column 1230, which can correspond to the ester/alcohol separation subsystem 1000 described with respect to FIG. 10. Column 1230 can be configured to separate methyl formate from methanol prior to hydrolysis. In specific embodiments, the separation column can be operated at a pressure of about 0.8 to 1.5 bar(a) to produce an overhead stream 1262 enriched in methyl formate, for example having a methyl formate concentration greater than about 90 wt %, and a bottoms stream enriched in methanol that is recycled to the esterification system 1220 as stream 1280.

The methyl formate-rich overhead stream 1262 from the separation column is supplied to a reactive distillation hydrolysis column 1240, which can correspond to the distillation column-based configuration described with respect to system 850 in FIG. 8. In specific embodiments, the ester-rich stream can be compressed and preheated, for example using a feed-product heat exchanger, to achieve a desired vapor fraction prior to entry into the column. Water is also supplied to the hydrolysis reactive distillation column. The hydrolysis column can be operated at a pressure of about 3 to 7 bara and can include a reaction zone containing a strong acid ion-exchange resin catalyst, for example Amberlyst 15DRY, together with associated rectifying and stripping sections.

Within the hydrolysis reactive distillation column 1240, methyl formate reacts with water to form formic acid and methanol while separation occurs within the column. The rectifying section of the column 1240 is designed with a sufficient number of theoretical stages to prevent reaction products from refluxing into the reaction zone, thereby ensuring that the products vaporize prior to reaching the reaction section. An overhead stream 1264 comprising unreacted methyl formate, methanol, and unreacted formic acid can be withdrawn and recycled to the methyl formate-methanol separation column 1230. A bottom product stream 1265 is withdrawn from the hydrolysis reactive distillation column 1240 under conditions selected to produce a concentrated formic acid stream, for example about 80 to 90 wt % formic acid and about 10 to 20 wt % water.

A comparative analysis was performed to evaluate the total energy demand, expressed as thermal or electrical energy, required to recover concentrated formic acid from a dilute aqueous feed containing approximately 5 wt % formic acid, as shown in Table 2. The esterification-hydrolysis processes described herein in Examples 1 and 2 demonstrate a substantially lower energy requirement compared to conventional recovery methods, including extractive distillation employing sulfolane as an extractant and liquid-liquid extraction utilizing 2-methyltetrahydrofuran as a solvent.

TABLE 2
Energy consumption comparison for concentration
of 5 wt % formic acid for Examples 1 and 2

| Separation Method | Total Energy |
|---|---|
| for 5 wt % Formic Acid | (MWh/tonne FA) |
| Extractive distillation | 37.36 |
| Liquid-liquid extraction | 13.61 |
| Esterification + hydrolysis reactor (Example 1) | 0.92 |
| Esterification + hydrolysis column (Example 2) | 1.34 |

### Example 3: CO Electrolyzer for Acetic and Propionic Acid Production with a Hydrolysis Reactor

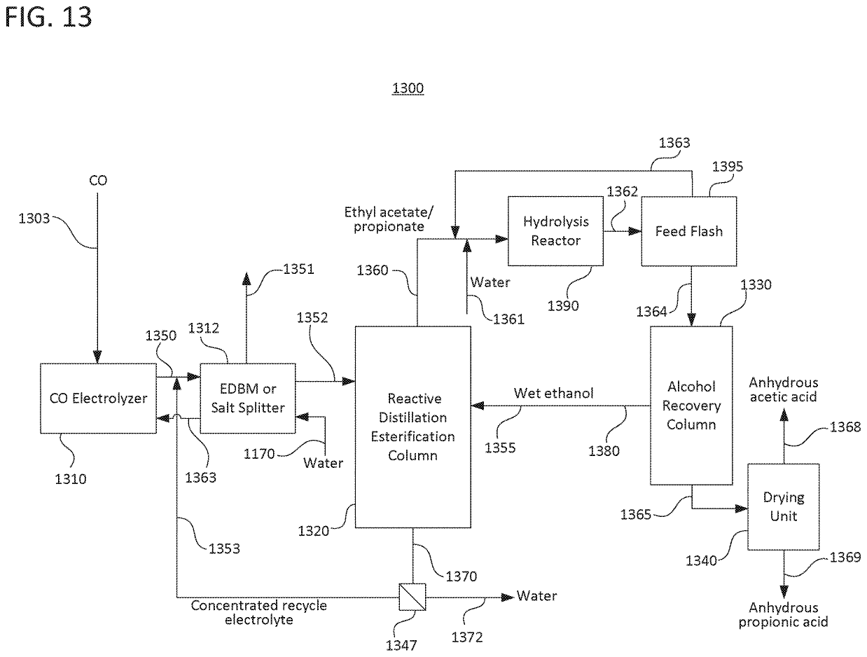

FIG. 13 shows a diagram of a system 1300 for recovering acetic acid and propionic acid from a CO-derived aqueous stream in accordance with specific embodiments of the inventions disclosed herein. In this example, the system 1300 represents a specific implementation of the acid recovery system 200 described with respect to FIG. 2, in which a CO electrolyzer system, a reactive distillation esterification column, and a hydrolysis and separation system are integrated to recover concentrated acetic and propionic acids.

In the embodiment illustrated in FIG. 13, a CO electrolyzer system 1310 takes CO stream 1303 as an input and produces a dilute aqueous carboxylic acid stream 1350 comprising acetate and propionate. In specific embodiments, the electrolyzer system 1310 and the EDBM or salt-splitter 1312 can correspond to the alkaline electrolyzer system 500 described with respect to FIG. 5. Stream 1350 can be acidified in the EDBM or salt-splitter 1312 to produce a carboxylic acid stream 1352 containing acetic and propionic acids. In this example, recycled electrolyte 1353 comprising potassium bisulfate and potassium sulfate are added to the EDBM feed stream 1350 to provide conductivity and acidic pH control in the downstream esterification column. A two-compartment EDBM can be used to produce potassium hydroxide to send back in stream 1363 to the CO electrolyzer. The acetate and propionate can be fully protonated to acetic acid and propionic acid at a discharge pH of 2-3. In some examples, the EDBM or salt-splitter 1312 may also have an output stream 1351. Dilute acid stream 1352 is sent to a reactive distillation esterification system 1320 together with an alcohol stream 1355. The esterification system 1320 can correspond to the reactive distillation systems 600 or 700 described with respect to FIG. 6 or 7. The esterification is run in an acid-excess regime. Within the esterification system 1320, the carboxylic acids react with ethanol to form ethyl acetate and ethyl propionate, which are removed as an ester-containing stream 1360. In specific embodiments, the esterification system can be operated under conditions selected to promote continuous removal of both esters as vapor while retaining non-volatile components within the column. A bottoms stream 1370 is removed containing excess acids (e.g., >10%) as well as potassium bisulfate and potassium sulfate. These are separated in a reverse osmosis (RO) unit 1347. A water stream 1372 from the RO unit 1347 can be routed to stream 1161, stream 1170, or other places in the system where makeup water is needed.

The ester-containing stream 1360 is supplied directly to a hydrolysis and separation system, which can correspond to the hydrolysis reactor-based configuration described with respect to system 800 in FIG. 8. Stream 1360 is combined with a water stream 1361 and introduced into a hydrolysis reactor 1390 in which ethyl acetate and ethyl propionate are hydrolyzed to acetic acid, propionic acid, and ethanol stream 1362. In specific embodiments, the hydrolysis reactor can include a strong acid ion exchange resin catalyst, for example Amberlyst 15DRY, and can be operated at elevated temperature and pressure, for example about 100° C. and about 2-5 bar(a).

The reaction mixture stream 1362 is supplied to a feed flash vessel 1395 configured to remove residual esters acetate and propionate. In specific embodiments, the pressure of the reaction mixture is reduced to promote vaporization of the esters, which are recovered as a vapor stream 1363 and recycled to the hydrolysis reactor 1390. A liquid stream 1364 comprising acetic acid, propionic acid, ethanol, and water is withdrawn from the feed flash vessel 1395 and supplied to an alcohol recovery column 1330.

The alcohol recovery column 1330 can separate ethanol from the hydrolysis mixture. An ethanol-rich overhead stream 1380, which can also contain water, is recovered and recycled to the reactive distillation esterification system 1320 as input wet ethanol stream 1355. A concentrated acetic and propionic acid stream 1365 is recovered from the bottoms of the column. In specific embodiments, the acetic and propionic acid product stream 1365 can have an acid concentration greater than about 50 wt %, for example about 60-85 wt %, and in specific embodiments about 85 wt %., and can be further processed if higher purity is desired. In specific embodiments, heat generated within the system can be integrated between process units as described with respect to system 200.

The acid stream 1365 can be further concentrated in an acid drying unit 1340, which can correspond to the acid drying system 900 described with respect to system 900 in FIG. 9. The configuration used here is much the same as in system 900, where separate streams of anhydrous acetic acid 1368 and anhydrous propionic acid 1369 are produced by the distillation column in the drying unit 1340.

In specific embodiments, one example of the material balance and process flow of the system in Example 3 is presented in Table 3. Other chemicals or combinations of chemicals (including those not listed or omitting chemicals listed in the table) may be present. Additionally, chemicals may be present in amounts or percentages other than those listed.

TABLE 3
Material Balance of Example 3

| | | | | Potassium | Acetic | Ethyl | Potassium | Propionic |
|---|---|---|---|---|---|---|---|---|
| | | K₂CO₃ | CO₂ | Acetate | Acid | Acetate | Propionate | Acid |
| Feed | kgmol/h | 0 | | 35 | | | 6 | |
| electrolyte | kg/h | 48 | | 3403 | | | 672 | |
| | wt % | 0.1% | | 4.7% | | | 0.9% | |
| Acid rich | kgmol/h | | 0 | | 39 | | | 7 |
| electrolyte | kg/h | | 15 | | 2315 | | | 493 |
| | wt % | | 0.0% | | 2.1% | | | 0.5% |
| Return KOH | kgmol/h | | | | | | | |
| Wet Ethanol | kgmol/h | | | | | | | |
| CO₂ | kgmol/h | | 0.3 | | | | | |
| Ethyl Acetate | kgmol/h | | | | | 35 | | |
| Acid Lean | kgmol/h | | | | 4 | | | 1 |
| Electrolyte | | | | | | | | |
| Concentrated | kgmol/h | | | | 4 | | | 1 |
| Recycle | | | | | | | | |
| Electrolyte | | | | | | | | |
| Recycle Ethyl | kgmol/h | | | | | 10 | | |
| Acetate | | | | | | | | |
| Water to | kgmol/h | | | | | | | |
| Hydrolysis | | | | | | | | |
| Hydrolysis | kgmol/h | | | | 35 | 10 | | 6 |
| Reactor | | | | | | | | |
| Product | | | | | | | | |
| Hydrolysis | kgmol/h | | | | 35 | | | 6 |
| Column Feed | | | | | | | | |
| Hydrolysis | kgmol/h | | | | 35 | | | 6 |
| Column | kg/h | | | | 2083 | | | 444 |
| Bottoms | wt % | | | | 70.50% | | | 15.00% |
| Recycle H₂SO₄ | kgmol/h | | | | | | | |
| | kg/h | | | | | | | |
| | wt % | | | | | | | |
| Dilute H₂SO₄ | kgmol/h | | | | | | | |
| | kg/h | | | | | | | |
| | wt % | | | | | | | |
| Product Acetic | kgmol/h | | | | | 34.7 | | |
| Acid | kg/h | | | | | 3055.6 | | |
| Product | kgmol/h | | | | | | | 6 |
| Propionic Acid | kg/h | | | | | | | 444 |

| | | Ethyl | | | | | | | |
|---|---|---|---|---|---|---|---|---|---|
| | | Propionate | Ethanol | K₂SO₄ | KHSO₄ | H₂SO₄ | KOH | Water | Total |
| Feed | kgmol/h | | | | | | 27 | 3742 | 3810 |
| electrolyte | kg/h | | | | | | 1509 | 67361 | 72993 |
| | wt % | | | | | | 2.1% | 92.3% | 100.0% |
| Acid rich | kgmol/h | | | 10 | 1 | | | 5757 | 5814 |
| electrolyte | kg/h | | | 1758 | 183 | | | 103633 | 108397 |
| | wt % | | | 1.6% | 0.2% | | | 95.6% | 100.0% |
| Return KOH | kgmol/h | | | | | | 73 | 4030 | 4103 |
| Wet Ethanol | kgmol/h | | 45 | | | | | 11 | 57 |
| CO₂ | kgmol/h | | | | | | | | 0 |
| Ethyl Acetate | kgmol/h | 6 | | | | | | 10 | 51 |
| Acid Lean | kgmol/h | | | 10 | 1 | | | 5798 | 5814 |
| Electrolyte | | | | | | | | | |
| Concentrated | kgmol/h | | | 10 | 1 | | | 2029 | 2045 |
| Recycle | | | | | | | | | |
| Electrolyte | | | | | | | | | |
| Recycle Ethyl | kgmol/h | 2 | | | | | | 3 | 15 |
| Acetate | | | | | | | | | |
| Water to | kgmol/h | | | | | | | 76 | 76 |
| Hydrolysis | | | | | | | | | |
| Hydrolysis | kgmol/h | 2 | 41 | | | | | | |
| Reactor | | | | | | | | | |
| Product | | | | | | | | | |
| Hydrolysis | kgmol/h | | 41 | | | | | 35 | 117 |
| Column Feed | | | | | | | | | |
| Hydrolysis | kgmol/h | | | | | | | 24 | 64 |
| Column | kg/h | | | | | | | 427 | 2954 |
| Bottoms | wt % | | | | | | | 14.40% | 100.00% |
| Recycle H₂SO₄ | kgmol/h | | | | | 29.1 | | 94.84 | 124 |
| | kg/h | | | | | 3962.9 | | 1707.1 | 5669.9 |
| | wt % | | | | | 69.90% | | 30.10% | 100.00% |
| Dilute H₂SO₄ | kgmol/h | | | | | 29.1 | | 118.5 | 147.7 |
| | kg/h | | | | | 3962.9 | | 2133.8 | 6096.7 |
| | wt % | | | | | 65.00% | | 35.00% | 100.00% |
| Product | kgmol/h | | | | | | | 0 | 34.7 |
| Acetic Acid | kg/h | | | | | | | 0 | 3055.6 |
| Product | kgmol/h | | | | | | | | 6 |
| Propionic Acid | kg/h | | | | | | | | 444 |

### Example 4: CO Electrolyzer for Acetic Acid Production with a Reactive Distillation Hydrolysis Column

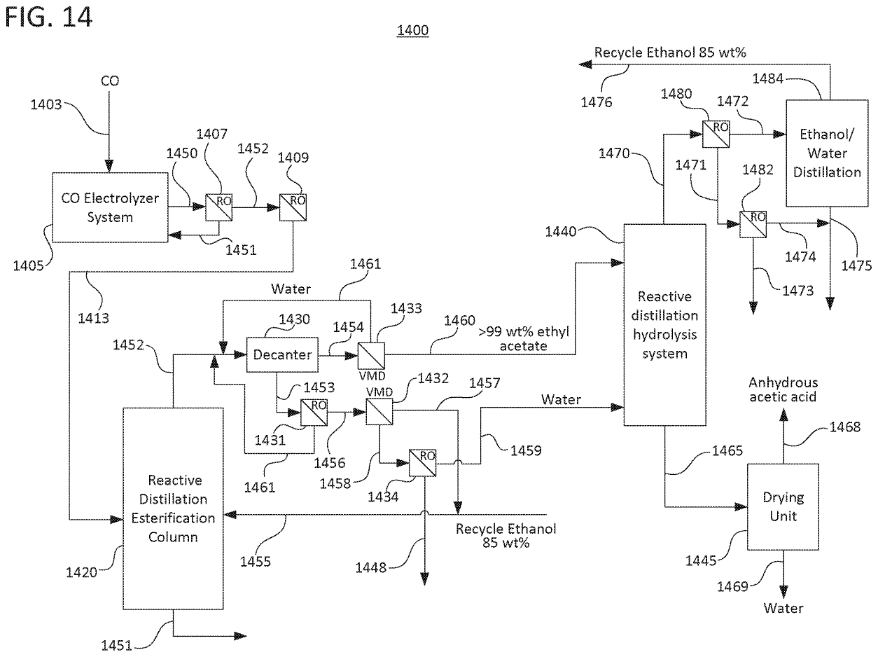

FIG. 14 shows a diagram of a system 1400 for recovering acetic acid from a CO-derived aqueous stream in accordance with specific embodiments of the inventions disclosed herein. In this example, the system 1400 represents a specific implementation of the acid recovery system 200 described with respect to FIG. 2, in which a CO electrolyzer system, a reactive distillation esterification column, and a hydrolysis and separation system are integrated to recover concentrated acetic acid.

In the embodiment illustrated in FIG. 14, a CO electrolyzer system 1405 takes CO stream 1403 as an input and produces a dilute aqueous carboxylic acid stream 1450 comprising acetic acid and is shown in schematic form. In specific embodiments, the electrolyzer system 1405 can correspond to the alkaline electrolyzer system 500 described with respect to FIG. 5. The CO electrolyzer system 1405 comprises a CO electrolyzer, an acid/base generator such as an EDBM or salt-splitter electrolyzer, and a degassing unit. Acid stream 1450 is sent to a loose membrane RO 1407 (e.g., using a cellulose acetate-based membrane), which selectively rejects potassium acetate and allows acetic acid to permeate. The potassium acetate stream 1451 is returned to the electrolyzer system 1405. The loose RO permeate stream 1452 is passed to a tight membrane RO 1409 (e.g., using a polyethyleneimine-based membrane). Permeate water from the tight membrane RO 1409 can be recycled back to the electrolyzer (not shown), while a relatively concentrated acetic acid stream 1413 is fed downstream. In this example, stream 1413 can be about 12 wt % acetic acid.

Acid stream 1413 is sent to a reactive distillation esterification column 1420 together with an alcohol stream 1455. The esterification column 1420 can correspond to the reactive distillation systems 600 or 700 described with respect to FIG. 6 or 7. The esterification is run in an acid-excess regime where much of the alcohol is reacted, which in this example is ethanol. The column 1420 operates at a vacuum (around 0.3 bara). The bottoms temperature is about 70° C., which allows electrolyzer byproduct heat to be used for reboiler duty. In specific embodiments, the column 1420 contains a reaction section which is filled with a strong acid ion exchange resin (e.g. AmberLyst 15DRY). The concentrated acetic acid stream 1413 from the tight RO concentrate is fed to the top of the reaction section. Recycle or makeup highly concentrated ethanol stream 1455 (e.g., 85 wt % ethanol) is routed to the middle of the reaction section where it reacts with the acetic acid from the tight RO concentrate feed under acidic conditions (pH 2-3) in an esterification reaction to produce ethyl acetate. The ethyl acetate exits the top of the column along with water vapor and a small amount of unreacted ethanol and acetic acid as stream 1452. Continuous stripping of the more volatile ethyl acetate allows nearly all of the ethanol to be converted to ethyl acetate. The continuous stripping and an excess of acetic acid versus ethanol in the feeds ensures that 90% or more of the ethanol is converted to ethyl acetate. This minimizes unreacted ethanol in the overhead condensate stream from the esterification column. Column 1420 is run in the acid-excess regime, so the excess acetic acid in the column remains in the column bottoms. The column bottoms stream 1451 can be routed to an esterification hydrophobic vacuum membrane distillation (VMD) system (not pictured) for recovery and recycle of the acetic acid.

In this example, the acetate is processed somewhat differently than in previously disclosed subsystem embodiments. In specific embodiments, the overhead condensate stream 1452 from the esterification column is routed to a decanter 1430 which separates the ethyl acetate (organic phase 1454) from the water, residual alcohol and residual acetic acid (aqueous phase 1453). The aqueous phase 1453 from the decanter is routed to a loose RO unit 1431 which selectively rejects the ethyl acetate from the decanter aqueous phase. The ethyl acetate is recycled as stream 1461. The water, ethanol, and acetic acid in the permeate 1456 are routed to a hydrophobic VMD 1432 which selectively vaporizes and condenses the ethanol from the remaining aqueous phase components. Low temperature (60-100° C.) heat from the electrolyzer system is used to provide heat to the hydrophobic VMD 1432. The recovered ethanol stream 1457 is recycled to the esterification reactive distillation system combined with ethanol stream 1455. The ethanol free aqueous phase stream 1458 from the VMD is fed to a tight RO 1434 to remove water and concentrate the remaining acetic acid to 12 wt % in the concentrate stream 1448. The 12 wt % acetic acid stream 1448 is recycled to the esterification reactive distillation system and can be combined with input stream 1413. The RO permeate 1459 (water) is recycled to the CO electrolyzer system 1440 or some can be recycled the CO electrolyzer system 1405 (not shown).

The ethyl acetate organic phase 1454 from the decanter 1430 containing 3 wt % water is routed to a hydrophilic VMD 1433 which removes the residual water from the ethyl acetate product stream 1460. At this point the ethyl acetate product stream 1460 can have a purity >99 wt %. Low temperature (60-100° C.) heat from the electrolyzer system is used to provide heat to the hydrophilic VMD. A water condensate stream 1461 is recycled to the decanter to prevent loss of ethyl acetate.

In specific embodiments, the dried ethylene acetate stream 1460 can be removed from the system as a final product. In specific embodiments, the ethylene acetate stream 1460 can be fed into a hydrolysis and separation system 1440 to produce a purified acetic acid product. System 1440 can correspond to the distillation column-based configuration described with respect to system 850 in FIG. 8. In specific embodiments, the reactive distillation hydrolysis column operates at 2-5 bara and consists of an upper reaction section filled with strong acid ion exchange resin (e.g. AmberLyst 15DRY), a middle entrainer section, and a bottom stripping section equipped with a reboiler. These sections may correspond to sections 871, 872, and 873 of the distillation column 870 in FIG. 8 respectively. The dry ethyl acetate stream 1460 from the hydrophilic VMD is routed to the entrainer section of the hydrolysis reactive distillation column. The bottoms product from the reaction section is fed to the top of the entrainer section. In the entrainer section, the feed ethyl acetate forms a lower boiling azeotrope with the residual water from the reaction section feed, allowing separation of the water from the product acetic acid. A stripping section below the entrainer section removes any residual lower boiling ethyl acetate/water azeotrope from the product acid stream using heat from a reboiler. The bottoms from the hydrolysis column is purified acetic acid (for example, 60-85 wt %). Recycling excess ethyl acetate to the hydrolysis column minimizes excess water required and serves as both an azeotropic entrainer and a reactant.

The ethyl acetate and water vapor stream from the entrainer section enters the bottom of the reaction section. Water from esterification is fed to the top of the reaction section. In the reaction section the ethyl acetate is hydrolyzed to produce ethanol and acetic acid. The ethanol, residual ethyl acetate and approximately 50% of the unreacted water leaves the top of the column as a condensate product 1470. The acetic acid and the remaining water leave the bottom of the reaction section and enter the entrainer section. An MVR compressor can be used to compress the product vapor from the top reaction section so that it can provide the heat for the reboiler. The product condensate stream from the reboiler is routed to a hydrolysis RO system.

In specific embodiments, a hydrolysis RO system uses two loose RO membranes in series. The first RO 1480 selectively rejects ethyl acetate to the concentrate stream 1471 and produces a concentrated alcohol and water stream 1472 (25 wt %) as a permeate. The concentrated alcohol permeate stream 1472 is routed to an ethanol water distillation column 1484. The concentrate stream 1471 from the first hydrolysis RO 1480 is mixed with water from the esterification section and routed to a second loose RO 1482. The second hydrolysis RO 1482 selectively rejects ethyl acetate to a concentrate stream 1473 and produces a dilute (10 wt %) ethanol and water stream 1474, which combines with the bottoms stream 1475 of the ethanol water distillation column 1484. The concentrate stream 1473 from the second hydrolysis RO can be recycled to the decanter 1430 in the esterification section.

The concentrated ethanol and water permeate stream 1472 from the first hydrolysis RO 1480 is fed to an atmospheric ethanol water distillation column 1484. The highly concentrated overhead ethanol stream 1476 from the distillation column (e.g., 85 wt % ethanol, 15 wt % water) can be compressed in an MVR compressor and condensed in the reboiler to provide heat to the ethanol column bottoms. The condensed overhead stream 1476 from the ethanol water distillation column is recycled to the esterification reactive distillation column system into stream 1455. The bottoms stream 1475 from the ethanol water distillation column (10 wt % ethanol, 90 wt % water) is routed to the hydrophilic VMD in the esterification section and may be combined with ethanol and water stream 1474.

In specific embodiments, the acid product stream 1465 can be optionally further concentrated in an acid drying unit 1445, which can correspond to the acid drying system 900 described with respect to system 900 in FIG. 9. The acid drying unit 1445 can produce a glacial grade purity (>99.85 wt %) output of acetic acid 1468 and a water stream 1469.

### Example 5: CO₂ Electrolyzer for Methyl Formate Production

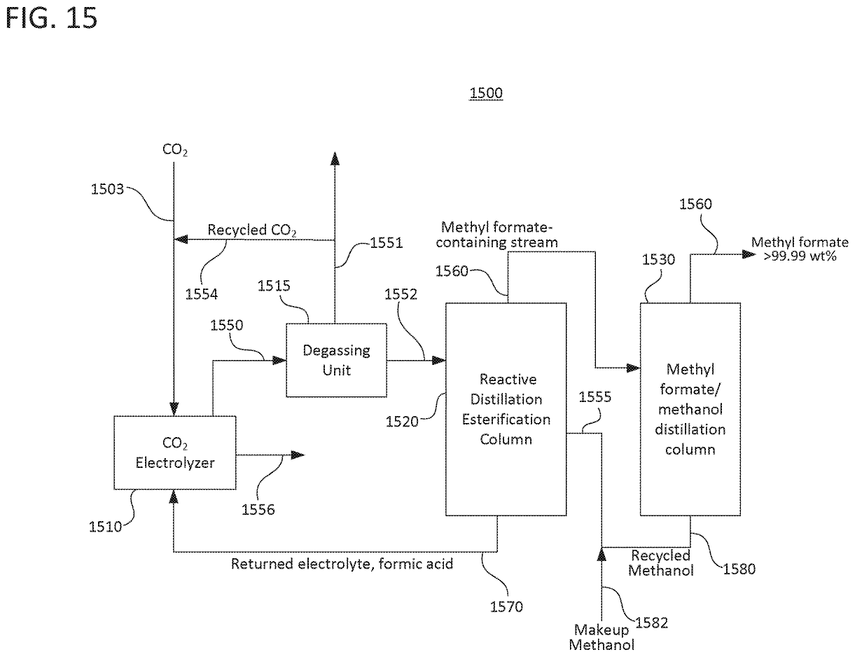

FIG. 15 provides a diagram of a system 1500 for recovering purified methyl formate from a dilute aqueous stream produced by a CO₂ electrolyzer system in accordance with specific embodiments of the inventions disclosed herein. In this example, the system 1500 represents a specific implementation of the ester recovery system 300 described with respect to FIG. 3, in which a CO₂ electrolyzer system, a reactive distillation esterification column, and an ester separation system are integrated to recover purified methyl formate.

In the embodiment illustrated in FIG. 15, a CO₂ electrolyzer system 1510 takes as input a feed stream 1503 of CO₂ and a recycled electrolyte and formic acid stream 1570 to produce a dilute aqueous stream 1550 comprising formic acid and stream 1556. In specific embodiments, CO₂ electrolyzer system 1510 corresponds to acidic electrolyzer 410 of FIG. 4. Stream 1550 can enter a degassing unit 1515 that produces a degassed aqueous stream 1552 comprising formic acid. The degassing unit 1515 also produces stream 1551. A portion of the stream 1551 containing CO₂ may be recycled as stream 1554 to combine with feed stream 1503. Stream 1552 is supplied to a reactive distillation esterification system 1520 together with a methanol stream 1555, where the carboxylic acid reacts with the alcohol to form a corresponding ester that is removed as an ester-containing stream 1560. In specific embodiments, the reactive distillation esterification system 1520 can correspond to the multi-section configuration of system 700 described with respect to FIG. 7 and can be operated in an alcohol-excess regime.

In specific embodiments, the esterification system 1520 comprises one or more columns that include a central reaction zone and two stripping zones. Within the esterification system 1520, methanol reacts with formic acid in the central reaction zone under acidic conditions within a pressure range, for example, of 0.9 to 3 bara, to produce methyl formate. The stripping zones can operate within a lower pressure range of 0.2 to 0.9 bara. The methyl formate-containing stream 1560 is removed with an overhead vapor stream comprising methyl formate and methanol, possibly along with some water, and a minor fraction of unreacted formic acid. In specific embodiments, methanol supplied to the system can comprise a recycled wet methanol stream 1580. In this example, no hydrolysis column is used to convert methyl formate back to formic acid, thus a makeup methanol 1582 stream can be combined with stream 1580 to form methanol input stream 1555. Stream 1555 can be divided into multiple portions and introduced at different locations within the esterification system to control reaction and separation behavior. For example, one portion of methanol can be introduced at the top of the reaction zone together with the incoming acid feed, while a second portion preheated by contact with the column's overhead vapor. This corresponds to the MVR configuration shown in FIG. 7. Stripping zones in the esterification system can recycle methanol to the methanol feed stream 1580.

The overhead vapor stream 1560 from the esterification system 1520 is supplied to a methyl formate-methanol separation column 1530, which can correspond to the ester/alcohol separation subsystem 1000 described with respect to FIG. 10. Column 1530 can be configured to separate methyl formate from methanol. In specific embodiments, the separation column can be operated at a pressure of about 0.8 to 1.5 bara and a temperature between 15-55° C. using a reflux ratio between 8 and 10.5. This produces an overhead stream with methyl formate concentration >99.99 wt %. Other separation column conditions could be chosen to adjust this if this high purity is not required. The corresponding bottoms stream 1580 enriched in methanol is recycled to the esterification system 1520.

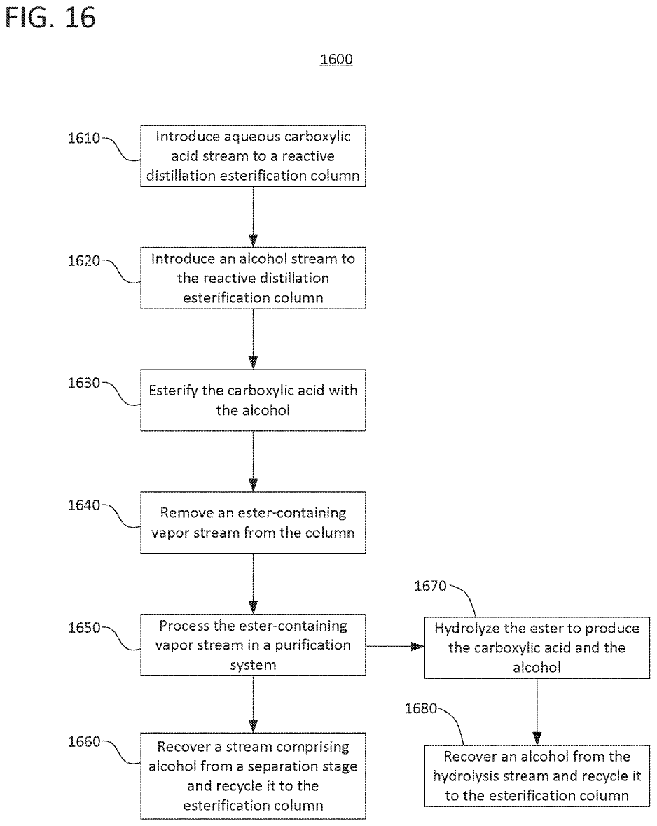

FIG. 16 provides a diagram of a process 1600 for producing a purified chemical product from an aqueous carboxylic acid-containing stream in accordance with specific embodiments of the inventions disclosed herein. Process 1600 begins with step 1610, where an aqueous carboxylic acid stream is introduced to a reactive distillation esterification column. In step 1620, the method includes introducing an aqueous feed stream comprising a carboxylic acid into a reactive distillation esterification column in step 1610. In step 1620, an alcohol is introduced into the reactive distillation esterification column. In step 1630, the carboxylic acid is esterified with the alcohol within the reactive distillation esterification column to form an ester. In step 1640, an ester-containing vapor stream is removed from the reactive distillation esterification column as the ester is formed. In step 1650, the ester-containing vapor stream is processed in a downstream purification system to produce a purified chemical product. In specific embodiments, additional optional steps can be included. In step 1660, alcohol recovered from downstream processing units can be recycled to the esterification column. In step 1670, the ester can be hydrolyzed to recreate the original carboxylic acid and alcohol. In step 1680, the alcohol hydrolysis product can also be recycled to the esterification column.

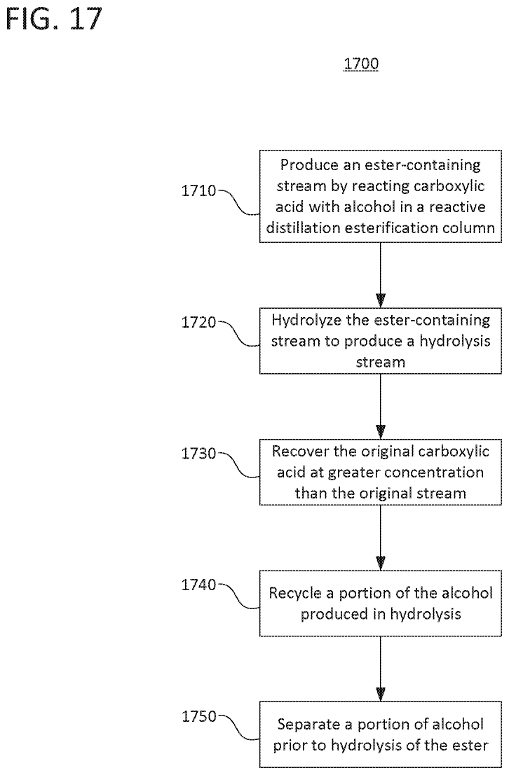

FIG. 17 provides a diagram of a process 1700 for purifying a carboxylic acid from an aqueous stream in accordance with specific embodiments of the inventions disclosed herein. Process 1700 begins with step 1710, where an ester-containing stream can be produced by reacting a carboxylic acid with an alcohol in a reactive distillation esterification column. In step 1720, at least a portion of the ester-containing stream is hydrolyzed to produce a hydrolysis stream comprising the carboxylic acid and the alcohol. In step 1730, a carboxylic acid product stream having a greater concentration than the aqueous carboxylic stream is recovered. In step 1740, at least a portion of the alcohol produced during hydrolysis is recycled to the reactive distillation esterification column. In specific embodiments, in step 1750, a portion of alcohol can be separated from the ester-containing stream prior to the hydrolysis step.

In certain embodiments, the reactive distillation esterification column described herein may be replaced, in whole or in part, by other reaction and/or separation technologies configured to perform a chemical transformation of a carboxylic acid-containing stream and to facilitate removal of a product stream having different separation characteristics than the original carboxylic acid. For example, the esterification reaction may be carried out in one or more stirred tank reactors, plug flow reactors, fixed-bed catalytic reactors, or loop reactors, optionally coupled with downstream or integrated separation units such as flash vessels, distillation columns, membrane separation units (including pervaporation, vapor permeation, or reverse osmosis), liquid-liquid extraction systems, phase separators, or decanters. In some embodiments, reaction and separation may be partially or fully integrated in hybrid systems, such as membrane reactors, reactive absorbers, reactive stripping units, or other intensified process configurations in which reaction and separation occur concurrently or in coordinated stages. In further embodiments, separation of the transformed product (e.g., an ester) from water, alcohol, and/or residual carboxylic acid may be achieved using non-distillation techniques, including selective membranes, adsorption systems, or solvent-based extraction processes, depending on the physical and chemical properties of the components. Accordingly, the use of a reactive distillation esterification column should be understood as one non-limiting example of a broader class of reaction-separation systems capable of enabling the transformation-assisted recovery or valorization of dilute carboxylic acid streams as described herein.

While the specification has been described in detail with respect to specific embodiments of the invention, it will be appreciated that those skilled in the art, upon attaining an understanding of the foregoing, may readily conceive of alterations to, variations of, and equivalents to these embodiments. These and other modifications and variations to the present invention may be practiced by those skilled in the art, without departing from the scope of the present invention, which is more particularly set forth in the appended claims.

What is claimed is:

1. A method for producing a purified chemical product from an aqueous carboxylic acid-containing stream, comprising:
introducing an aqueous feed stream comprising a carboxylic acid into a reactive distillation esterification column;
introducing an alcohol stream into the reactive distillation esterification column;
esterifying the carboxylic acid with the alcohol within the reactive distillation esterification column to form an ester;
removing an ester-containing vapor stream from the reactive distillation esterification column as the ester is formed; and
processing the ester-containing vapor stream in a downstream purification system to produce the purified chemical product.

2. The method of claim 1 wherein the aqueous feed stream comprises less than about 20 wt % of the carboxylic acid.

3. The method of claim 1 wherein the ester-containing vapor stream is continuously removed from the reactive distillation esterification column as the ester is formed.

4. The method of claim 1 further comprising recovering a stream comprising alcohol from a downstream separation stage and recycling the stream to the reactive distillation esterification column.

5. The method of claim 1 wherein the alcohol is introduced in molar excess relative to the carboxylic acid.

6. The method of claim 5 wherein the alcohol is introduced at a molar ratio greater than about 1.2:1 relative to the carboxylic acid.

7. The method of claim 1 wherein the carboxylic acid is introduced in molar excess relative to the alcohol.

8. The method of claim 7 wherein the molar excess of carboxylic acid reduces formation of azeotropic mixtures during separation of the ester.

9. The method of claim 7 wherein less than about 80% of the carboxylic acid is converted to the ester within the reactive distillation esterification column.

10. The method of claim 1 further comprising separating the ester-containing vapor stream to produce a purified ester product.

11. The method of claim 10 wherein the alcohol comprises methanol and the purified ester product comprises methyl formate.

12. The method of claim 10 wherein separating the ester-containing vapor stream to produce the purified ester product includes a rectification column configured to produce an ester having a purity greater than about 99 wt %.

13. The method of claim 1 further comprising hydrolyzing the ester to produce a hydrolysis stream comprising the carboxylic acid and the alcohol.

14. The method of claim 13 further comprising recycling at least a portion of the alcohol in the hydrolysis stream to the reactive distillation esterification column.

15. The method of claim 1 wherein the carboxylic acid is selected from C1-C4 carboxylic acids.

16. The method of claim 1 wherein the aqueous feed stream comprises a carboxylic acid produced by a carbon oxide electrolyzer system.

17. The method of claim 1 wherein non-volatile constituents in the aqueous feed stream remain in a bottoms stream of the reactive distillation esterification column while the ester is removed in the ester-containing vapor stream.

18. A system for producing a purified chemical product from an aqueous carboxylic acid-containing stream, comprising:
a reactive distillation esterification column comprising:
an aqueous feed inlet configured to receive an aqueous stream comprising a carboxylic acid, an alcohol inlet configured to receive an alcohol, a reaction zone within the column in which the carboxylic acid reacts with the alcohol to form an ester, an overhead vapor outlet configured to withdraw an ester-containing vapor stream, and a bottoms outlet configured to discharge a bottoms stream; and
a downstream purification system fluidly connected to the overhead vapor outlet that receives the ester-containing vapor stream and produces the purified chemical product.

19. The system of claim 18 wherein the purification system comprises a hydrolysis column fluidly connected to the overhead vapor outlet whereby the ester is converted back into the carboxylic acid and the alcohol.

20. The system of claim 18 wherein the purification system comprises a separation column fluidly connected to the overhead vapor outlet and configured to separate the ester-containing vapor stream to produce a purified ester product.

21. The system of claim 18 further comprising a carbon oxide electrolyzer configured to produce the aqueous feed stream comprising the carboxylic acid and to supply the aqueous feed stream to the aqueous feed inlet of the reactive distillation esterification column.

22. The system of claim 21 further comprising an electrodialysis bipolar membrane unit or a salt-splitting electrolyzer fluidly connected to receive an output stream from the carbon oxide electrolyzer when the carbon oxide electrolyzer is operated with an alkaline electrolyte and produces a carboxylate-containing stream;
wherein, the electrodialysis bipolar membrane unit or the salt-splitting electrolyzer converts the carboxylate to a carboxylic acid and supplies the aqueous feed stream comprising the carboxylic acid to the reactive distillation esterification column.

23. The system of claim 22 further comprising an ion exchange resin bed, where the electrodialysis bipolar membrane unit or the salt-splitting electrolyzer are fluidly connected with the ion exchange resin bed.

24. The system of claim 21 wherein a bottoms stream from the reactive distillation esterification column comprising water and/or electrolyte is supplied to the carbon oxide electrolyzer or an associated electrolyte circulation system.

25. A method for purifying a carboxylic acid from an aqueous stream, comprising:
producing an ester-containing stream by reacting an aqueous carboxylic acid stream with an alcohol stream in a reactive distillation esterification column;
hydrolyzing at least a portion of the ester-containing stream to produce a hydrolysis stream comprising the carboxylic acid and the alcohol;
recovering a carboxylic acid product stream having a greater concentration than the aqueous carboxylic acid stream; and
recycling at least a portion of the alcohol produced during hydrolysis to the reactive distillation esterification column.

26. The method of claim 25 wherein the hydrolysis is performed in a hydrolysis reactor or a hydrolysis reactive distillation column.

27. The method of claim 25 wherein the recovered carboxylic acid stream contains greater than about 50 wt % carboxylic acid.

28. The method of claim 25 wherein the aqueous stream comprises less than about 20 wt % of the carboxylic acid.

29. The method of claim 25 wherein the ester-containing stream is produced by removing an ester-containing vapor stream from a reactive distillation esterification column as the ester is formed.

30. The method of claim 25 further comprising separating at least a portion of alcohol from the ester-containing stream prior to hydrolyzing the ester.
